(19) United States
(12) Patent Application Publication
(10) Pub. No.: US 2024/0351990 A1
(43) Pub. Date: Oct. 24, 2024

(54) PROCESS FOR PREPARING 5-FLUORO-4-IMINO-3-METHYL-1-(TOLUENE-4- SULFONYL)-3,4-DIHYDRO-1H-PYRIMIDIN-2-ONE

(71) Applicant: Adama Makhteshim Ltd., Beer Sheva (IL)

(72) Inventor: Gal Suez, Dimona (IL)

(73) Assignee: Adama Makhteshim Ltd., Beer Sheva (IL)

(21) Appl. No.: 18/692,018

(22) PCT Filed: Sep. 15, 2022
(86) PCT No.: PCT/IB2022/058732

§ 371 Date: Mar. 14, 2024

Related U.S. Application Data
(60) Provisional application No. 63/244,562, filed on Sep. 15, 2021.

(51) Int. Cl. C07D 239/47

(52) U.S. Cl. CPC C07D 239/47

(57) ABSTRACT

The present invention provides a process for obtaining 5-fluoro-4-imino-3-methyl-1-(phenyl-4-sulfonyl)-3, 4-dihydro-1H-pyrimidin-2-one having formula (I):

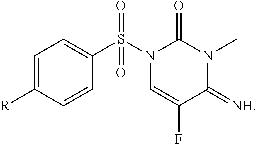

The present invention also provides a method for isolating a compound having the formula (I). The present invention also provides a monomethylsulfate salt of the compound having formula (I). The present invention also provides a method for crystallizing or recrystallizing a compound having formula (I) using an anti-solvent. The present invention also provides a method of isolating a compound having formula (II).

Throughout this application various publications are referenced. The disclosures of these documents in their entireties are hereby incorporated by reference into this application in order to more fully describe the state of the art to which this invention pertains.

### TECHNICAL FIELD

The present subject matter relates to an efficient procedure for obtaining 5-fluoro-4-imino-3-methyl-1-(toluene-4-sulfonyl)-3, 4-dihydro-1H-pyrimidin-2-one.

### BACKGROUND

The compound 5-fluoro-4-imino-3-methyl-1-(toluene-4-sulfonyl)-3, 4-dihydro-1H-pyrimidin-2-one has the structure:

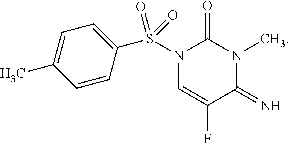 [SMILES: Cc1ccc(S(=O)(=O)n2cc(F)c(=N)n(C)c2=O)cc1]

5-fluoro-4-imino-3-methyl-1-(toluene-4-sulfonyl)-3, 4-dihydro-1H-pyrimidin-2-one is a systemic fungicide which provides control of variety of pathogens in economically important crops including, but not limited to, the causal agent of leaf blotch in wheat, Septoria tritici (SEPTTR).

Different manufacturing processes are known from the literature, including those described in WO 2015/103144 and WO 2015/103142.

There is a need to develop a more efficient synthesis pathway in terms of costs, yield, conversion and purity.

### SUMMARY OF THE INVENTION

The present invention provides a process for obtaining 5-fluoro-4-imino-3-methyl-1-(phenyl-4-sulfonyl)-3, 4-dihydro-1H-pyrimidin-2-one having formula (I):

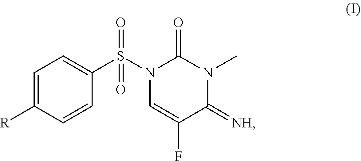

comprising:
(1) preparing the compound having formula (I) by reacting a compound having the formula (II)

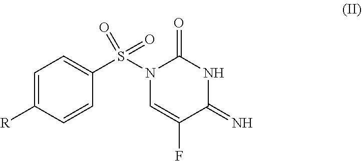

with dimethylsulphate (DMS), wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃, and
(2) isolating the compound having formula (I) from the reaction mixture by adding an aqueous basic solution to the reaction mixture and obtaining the compound having formula (I), wherein the process comprises use of at least one water
   i) step (1) is conducted in the presence of at least one water-immiscible solvent, and/or
   ii) at least one water-immiscible solvent is added to the reaction mixture after step (1).

The present invention also provides a method for isolating a compound having formula (I) from a mixture comprising the compound having formula (I), wherein the method comprises (i) preparing a multi-phase system comprising the compound having formula (I), a water-immiscible solvent and water, and (ii) obtaining and isolating solids of the compound having formula (I) from the multi-phase system.

The present invention also provides a monomethylsulfate salt of the compound having formula (I).

The present invention also provides a method for crystallizing or recrystallizing a compound having formula (I), comprising (i) preparing a solution comprising a compound having formula (I) and a solvent, and (ii) contacting the solution with an anti-solvent.

The present invention also provides use of an anti-solvent to crystalize or recrystallize a compound having formula (I) from a solution thereof.

The present invention also provides a method for isolating a compound having formula (II) from a mixture comprising the compound having formula (II), wherein the method comprises (i) adding a protic solvent to the mixture to precipitate the compound having formula (II) from the mixture, and (ii) collecting the precipitated compound of formula (II).

### DETAILED DESCRIPTION OF THE INVENTION

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one of skill in the art, that in some specific instances, an embodiment can alternatively be described using the language “consisting essentially of” or “consisting of”. In each such instance, the terms “comprising,” “consisting essentially of,” and “consisting of” are intended to have the same meaning as each such term would have when used as the transition phrase of a patent claim.

In an embodiment, use of the term “about” herein specifically includes ±10% from the indicated values in the range. By way of example, about 15% therefore includes 13.5%, 13.6%, 13.7%, etc. up to 16.5%. Use of the term “about” herein more specifically includes ±1% from the indicated values in the range. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

In addition, the endpoints of all ranges directed to the same component or property herein are inclusive of the endpoints, are independently combinable, and include all intermediate points and ranges. Where a range is given in the specification it is understood that the range includes all integers and 0.1 units within that range, and any sub-range thereof. For example, a range of “2-18%” is a disclosure of 2.0%, 2.1%, 2.2%, 2.3% etc. up to 18%.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₅ alkyl, C₃-C₈ alkyl, C₄-C₈ alkyl and so on.

As used herein, “alkoxy” represents an alkyl group as described above attached through an oxygen bridge.

As used herein, “Ph” is referring to phenyl group.

As used herein, the term “soluble” means when 1 g of substance is dissolved in the approximate volume of 100 ml.

In some embodiments, the term “water immiscible” when used in relation to a solvent means that the solvent does not completely mix with water to form a one phase solution.

As used in describing the tosylation step, the term “polar solvent” refers to solvent which has a dielectric constant equal to or above 20. As used in the remaining application, including in describing the sulfonation step other than tosylation step, the alkylation step and the isolation step, the term “polar solvent” has the meaning commonly understood by one of skill in the art to which this subject matter pertains, and includes, but is not limited to, solvent which has a dielectric constant equal to or above 20.

The polar solvent used in each of the sulfonation step, the alkylation step and isolation step may be the same or different. When the sulfonation step is a tosylation step, the polar solvent has a dielectric constant equal to or above 20.

As used herein, the term “formula (II)” refers to the following structure:

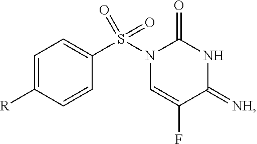

wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃, and tautomers thereof, including but not limited to enamine tautomers thereof.

As used herein, the term “formula (IIai)” refers to the following structure:

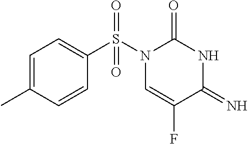 [SMILES: Cc1ccc(S(=O)(=O)n2cc(F)c(=N)[nH]c2=O)cc1]

and tautomers thereof, including but not limited to an enamine tautomer thereof. For example, the compound having formula (IIai) includes both of the following compounds:

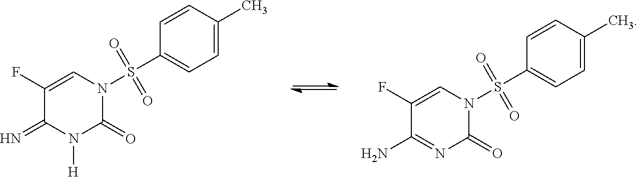 [SMILES: Cc1ccc(S(=O)(=O)n2cc(F)c(=N)[nH]c2=O)cc1 ; Cc1ccc(S(=O)(=O)n2cc(F)c(N)nc2=O)cc1]

The present invention provides a process for obtaining 5-fluoro-4-imino-3-methyl-1-(phenyl-4-sulfonyl)-3, 4-dihydro-1H-pyrimidin-2-one having formula (I):

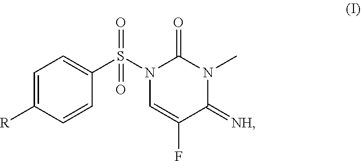

comprising:
(1) preparing the compound having formula (I) by reacting a compound having the formula (II)

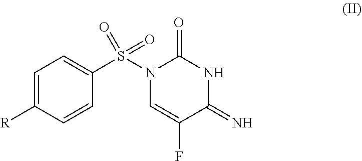

with dimethylsulphate (DMS), wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃, and
(2) isolating the compound having formula (I) from the reaction mixture by adding an aqueous basic solution to the reaction mixture and obtaining the compound having formula (I),
wherein the process comprises use of at least one water immiscible solvent, wherein:
   i) step (1) is conducted in the presence of at least one water-immiscible solvent, and/or
   ii) at least one water-immiscible solvent is added to the reaction mixture after step (1).

In some embodiments, R is alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃.

In some embodiments, the compound having the formula (II) is (IIa) wherein R is alkyl.

In some embodiments, the compound having the formula (IIa) is (IIai) wherein R is methyl.

In some embodiments, the compound having the formula (I) is (Ia) wherein R is alkyl.

In some embodiments, the compound having the formula (Ia) is (Iai) wherein R is methyl.

In some embodiments, the process forms a multi-phase system. In some embodiments, at the end of step 2, a multi-phase system is obtained.

In some embodiments, the multi-phase system comprises an organic phase and a water phase. In some embodiments, the multi-phase system is a slurry mixture comprising solids. The type of multi-phase system formed depends on the volume of the water-immiscible solvent and/or the temperature.

In some embodiments, wherein the multi-phase system comprises an organic phase and a water phase, the organic phase comprises the water-immiscible solvent. In some embodiments, wherein the multi-phase system comprises an organic phase and a water phase, the process comprises heating the multi-phase system for dissolution of the compound having formula (I) in the organic phase. In some embodiments, the process comprises heating the multi-phase system up to 80° C. for dissolution of the compound having formula (I) in the organic phase.

In some embodiments, wherein the multi-phase system comprises an organic phase and a water phase, the process for isolating the compound having formula (I) from the reaction mixture comprises separating the organic phase from the water phase, crystallizing the compound having formula (I) from the organic phase, and filtering the crystals.

For example, the compound having formula (I) may be isolated from the reaction mixture in according with the process described herein as route 2.

In some embodiments, step (1) is conducted in the presence of at least one water-immiscible solvent and the reaction mixture comprises the compound having formula (I), DMS and at least one water immiscible solvent.

In some embodiments, the water immiscible solvent is added after step (1) and before step (2) and the reaction mixture comprises the compound having formula (I), DMS and at least one water immiscible solvent.

In some embodiments, wherein the reaction mixture comprises the compound having formula (I), DMS and at least one water immiscible solvent, step (2) for isolating the compound having formula (I) from the reaction mixture comprises (i) washing the mixture with an aqueous basic solution to form an organic phase and a water phase, (ii) separating the organic phase from the water phase, and (iii) crystallizing the compound having the formula (I) from the organic phase and filtering the crystals.

In some embodiments, the process comprises (1) preparing the compound having formula (I) by reacting the compound having the formula (II) with dimethylsulphate (DMS) in the presence of at least one water immiscible solvent, and (2) isolating the compound having formula (I) from the reaction mixture by (i) washing the reaction mixture with an aqueous basic solution to from an organic phase and a water phase, (ii) separating the organic phase from the water phase, and (iii) crystalizing the compound having the formula (I) from the organic phase and filtering the crystals.

In some embodiments, the process comprises (1) preparing the compound having formula (I) by reacting the compound having the formula (II) with dimethylsulphate (DMS), (2) adding at least one water immiscible solvent to the reaction mixture, and (3) isolating the compound having formula (I) from the reaction mixture by (i) washing the reaction mixture with an aqueous basic solution to form an organic phase and a water phase, (ii) separating the organic phase from the water phase, and (iii) crystalizing the compound having the formula (I) from the reaction mixture and filtering the crystallized solid, or
(a) (1) preparing the compound having formula (I) by reacting the compound having the formula (II) with dimethylsulphate (DMS), and (2) isolating the compound having formula (I) from the reaction mixture by (i) adding at least one water immiscible solvent and an aqueous basic solution to the reaction mixture to form an organic phase and a water phase, (ii) separating the organic phase from the water phase, and (iii) crystalizing the compound having the formula (I) and filtering the crystallized solid.

In some embodiments, the aqueous basic solution comprises DABCO, TBAB, NaOH, K₂CO₃, KHCO₃, Na₂CO₃, Et₃N, NaOMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof. In some embodiments, the aqueous basic solution is an aqueous solution of a base selected from the group consisting of K₂CO₃, KHCO₃, Na₂CO₃, NaHCO₃, K₂CO₃ NH₄OH, NaOH, and any combination thereof. In some embodiments, the aqueous basic solution is an aqueous solution of K₂CO₃.

In some embodiments, the concentration of the base in the aqueous basic solution is 2-18% based on the total weight (w/w). In some embodiments, the concentration of the base in the aqueous basic solution is about 15% based on the total weight (w/w).

In some embodiments, the compound having the formula (I) is dissolved in the water immiscible solvent or a mixture of a water immiscible solvent with a water miscible solvent to obtain an organic solution. In some embodiments, the compound having the formula (I) in salt form is dissolved in the water immiscible solvent or a mixture of a water immiscible solvent with a water miscible solvent to obtain an organic solution.

In some embodiments, the water immiscible solvent is polar. In some embodiments, the water immiscible solvent is non-polar. In some embodiments, the water immiscible solvent is an organic polar solvent.

In some embodiments, the water immiscible solvent includes but not limited to cyclopentylmethylether (CPME), methyl tetrahydrofuran (MeTHF), DCM, toluene, anisole or any combination thereof. In some embodiments, the water immiscible solvent is selected from the group consisting of CPME, MeTHF, DCM, toluene, anisole, and any combination thereof.

In some embodiments, the water immiscible solvent is selected from the group consisting of MeTHF, CPME, toluene, anisole, and any mixture thereof.

In some embodiments, the water immiscible solvent is CPME. In some embodiments, the water immiscible solvent is MeTHF. In some embodiments, the water immiscible solvent is DCM. In some embodiments, the water immiscible solvent is toluene. In some embodiments, the water immiscible solvent is anisole.

In some embodiments, the compound having the formula (I) is crystallized from the organic phase by concentrating the organic phase. In some embodiments, the compound having the formula (I) is crystallized from the organic phase by adding an anti-solvent. In some embodiments, the compound having the formula (I) is crystallized from the organic phase by seeding.

In some embodiments, the anti-solvent is a C5-C11 alkane. In some embodiments, the anti-solvent is hexane. In some embodiments, the anti-solvent is heptane.

In some embodiments, the compound having formula (I) in the mixture is in salt form.

In some embodiments, the process for isolating the compound having formula (I) comprises (1) washing of an organic solution comprising a polar water immiscible solvent and a mixture of compound (I) and DMS with 2-18% w/w of aqueous basic solution, (2) separating the organic phase from the water phase, and (3) concentrating the organic phase and filtering the precipitated solid.

In some embodiments, the process for isolating the compound having formula (I) from a mixture comprising the compound having formula (I) and DMS comprises (1) dissolving the mixture comprising the compound having formula (I) and DMS in an organic polar solvent to obtain an organic solution, (2) washing the organic solution obtained from (1) with 2-18% w/w of aqueous basic solution, (3) separating the organic phase from the water phase, and (4) concentrating the organic phase and filtering the precipitated solid.

In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20.

In some embodiments, the organic polar solvent is an organic polar water immiscible solvent.

In some embodiments, the organic water immiscible solvent has a dielectric constant less than 20.

In some embodiments, organic water immiscible solvent includes but is not limited to methyl tetrahydrofuran (MeTHF), cyclopentylmethylether (CPME), and a mixture thereof.

In some embodiments, the organic polar solvent is selected from the group consisting of DMA, CPME, MeTHF, DMA, DMF, DCM and any combination thereof.

In some embodiments, the organic polar solvent is selected from the group consisting of CPME, MeTHF, DCM and any combination thereof.

In some embodiments, the organic polar solvent is DMA. In some embodiments, the organic polar solvent is CPME. In some embodiments, the organic polar solvent is MeTHF.

In some embodiments, the compound having formula (I) is a compound having formula (Ia) wherein R is alkyl.

In some embodiments, the compound having formula (Ia) is a compound having formula (Iai) wherein R is methyl.

In some embodiments, the organic phase is cooled prior to and/or during the filtration. In some embodiments, the organic phase is cooled to 0-5° C.

In some embodiments, the organic polar solvent is added after the reaction to obtain the compound having the formula (I). In some embodiments, the CPME is added after the reaction to obtain the compound having the formula (I).

In some embodiments, a partial amount of the organic polar solvent is present from the reaction to obtain the compound having formula (I) and optionally additional amount of the organic polar solvent is added before isolation of the compound having formula (I). In some embodiments, a partial amount of the CPME is present from the reaction to obtain the compound having formula (I) and optionally an additional amount of CPME is added before isolation of the compound having formula (I).

In some embodiments, the compound having the formula (I) in the mixture before the isolation step is in salt form.

In some embodiments, the non-polar solvent is a non-polar water immiscible solvent.

In some embodiments, the non-polar water immiscible solvent dissolves the compound of formula (I).

In some embodiments, the water immiscible solvent includes but is not limited to ether-based solvent, aromatic solvent such as CPME, THE, anisole, toluene, and any mixture thereof.

In some embodiments, the non-polar solvent is anisole. In some embodiments, the non-polar solvent is toluene.

In some embodiments, an anti-solvent is further added.

In some embodiments, the anti-solvent is added parallel to the to the water immiscible solvent.

In some embodiments, the anti-solvent is added dropwise.

In some embodiments, the crystallization is conducted at temperature less than 0° C.

In some embodiments, the anti-solvent is a C5-C11 alkane. In some embodiments, the anti-solvent is hexane. In some embodiments, the anti-solvent is heptane.

In some embodiments, the mixture of solvent and anti-solvent are anisole and hexane.

In some embodiments, the mixture of solvent and anti-solvent are toluene and hexane.

In some embodiments, the aqueous basic solution comprises DABCO, TBAB, NaOH, K₂CO₃, KHCO₃, Na₂CO₃, Et₃N, NaoMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaoMe, NaOEt and any combination thereof.

In some embodiments, the aqueous basic solution is an aqueous solution of K₂CO₃.

In some embodiments, the compound having formula (I) is a compound having formula (Ia) wherein R is alkyl.

In some embodiments, the compound having formula (Ia) is a compound having formula (Iai) wherein R is methyl.

In some embodiments, the organic phase is cooled prior to and/or during the filtration. In some embodiments, the organic phase is cooled to 0-5° C.

In some embodiments, the crystallization is done by concentration of the solvent.

In some embodiments, the crystallization is done with crystal seeding.

In some embodiments, the mixture is seeded with 0.1-1% of the compound of formula (I).

In some embodiments, the water immiscible solvent is added after the reaction to obtain the compound having the formula (I). In some embodiments, anisole and hexane are added after the reaction to obtain the compound having the formula (I).

In some embodiments, a partial amount of the water immiscible solvent is present from the reaction to obtain the compound having formula (I) and optionally additional amount of the water immiscible solvent is added before isolation of the compound having formula (I). In some embodiments, a partial amount of the anisole is present from the reaction to obtain the compound having formula (I) and optionally an additional amount of anisole optionally with hexane are added before isolation of the compound having formula (I).

In some embodiments, the process for isolating the compound having formula (I) comprises contacting the mixture comprising the compound having formula (I) with a water-immiscible solvent or a mixture of solvents comprising at least one water immiscible solvent and water, separating the organic phase, crystalizing the compound having the formula (I), and (3) filtering the crystals.

In some embodiments, wherein the multi-phase system is a slurry comprising solids, the process for isolating the compound having formula (I) form the reaction mixture comprises filtering the precipitated solids.

For example, the compound having formula (I) may be isolated from the reaction mixture in according with the process described herein as route 3.

In some embodiments, step (1) is conducted in the presence of at least one water-immiscible solvent and the reaction mixture comprises the compound having formula (I), DMS, and at least one water immiscible solvent, step (2) for isolating the compound having formula (I) from the reaction mixture comprises (i) washing the mixture with an aqueous basic solution to obtain a slurry mixture comprising solids, and (ii) filtering the precipitated solids.

In some embodiments, the water immiscible solvent is added after step (1) and the reaction mixture comprises the compound having formula (I), DMS, and at least one water immiscible solvent, step (2) for isolating the compound having formula (I) from the reaction mixture comprises (i) washing the mixture with an aqueous basic solution to obtain a slurry mixture comprising solids, and (ii) filtering the precipitated solids.

In some embodiments, step (1) is conducted in the presence of at least one additional solvent.

In some embodiments, wherein (a) step (1) is conducted in the presence of at least one additional solvent, (b) step (1) is conducted in the presence of at least one water-immiscible solvent and (c) the reaction mixture comprises the compound having formula (I), DMS, at least one water immiscible solvent, and at least one additional solvent, step (2) for isolating the compound having formula (I) comprises (i) washing the mixture with an aqueous basic solution to obtain a slurry mixture comprising solids, and (ii) filtering the precipitated solids.

In some embodiments, wherein (a) step (1) is conducted in the presence of at least one additional solvent, (b) the water immiscible solvent is added after step (1), and (c) the reaction mixture comprises the compound having formula (I), DMS, at least one water immiscible solvent, and at least one additional solvent, step (2) for isolating the compound having formula (I) comprises (i) washing the mixture with an aqueous basic solution to obtain a slurry mixture comprising and solids, (ii) filtering the precipitated solids.

In some embodiments, at least one additional solvent is added after step (1) and before step (2).

In some embodiments, wherein (a) at least one additional solvent is added after step (1), (b) step (1) is conducted in the presence of at least one water-immiscible solvent and (c) the reaction mixture comprises the compound having formula (I), DMS, at least one water immiscible solvent, and at least one additional solvent, step (2) for isolating the compound having formula (I) comprises (i) washing the mixture with an aqueous basic solution to obtain a slurry mixture comprising solids, and (ii) filtering the precipitated solids.

In some embodiments, wherein (a) at least one additional solvent is added after step (1), (b) the water immiscible solvent is added after step (1), and (c) the reaction mixture comprises the compound having formula (I), DMS, at least one water immiscible solvent, and at least one additional solvent, step (2) for isolating the compound having formula (I) comprises (i) washing the mixture with an aqueous basic solution to obtain a slurry mixture comprising solids, and (ii) filtering the precipitated solids.

In some embodiments, step (2) is conducted in the presence of at least one additional solvent.

In some embodiments, wherein step (1) is conducted in the presence of at least one water immiscible solvent and the reaction mixture comprises the compound having formula (I), DMS, and at least one water immiscible solvent, step (2) for isolating the compound having formula (I) comprises (i) washing the mixture with an aqueous basic solution and at least one additional solvent to obtain a slurry mixture comprising solids, and (ii) filtering the precipitated solids.

In some embodiments, wherein the water immiscible solvent is added after step (1) and the reaction mixture comprises the compound having formula (I), DMS, and at least one water immiscible solvent, step (2) for isolating the compound having formula (I) comprises (i) washing the mixture with an aqueous basic solution and at least one additional solvent to obtain a slurry mixture comprising solids, and (ii) filtering the precipitated solids.

In some embodiments, the process comprises (1) preparing the compound having formula (I) by reacting the compound having the formula (II) with dimethylsulphate (DMS) in the presence of at least one water immiscible solvent and at least one additional solvent, and (2) isolating the compound having formula (I) from the reaction mixture by (i) washing the reaction mixture with an aqueous basic solution to obtain a slurry mixture comprising precipitated solids, and (ii) filtering the precipitated solids.

In some embodiments, the process comprises (1) preparing the compound having formula (I) by reacting the compound having the formula (II) with dimethylsulphate (DMS), (2) adding at least one water immiscible solvent and at least one additional solvent to the reaction mixture, and (3) isolating the compound having formula (I) from the reaction mixture by (i) washing the reaction mixture with an aqueous basic solution to obtain a slurry mixture containing precipitated solids, and (ii) filtering the precipitated solids.

In some embodiments, the process comprises (1) preparing the compound having formula (I) by reacting the compound having the formula (II) with dimethylsulphate (DMS), (2) adding at least one water immiscible solvent to the reaction mixture, and (3) isolating the compound having formula (I) from the reaction mixture by (i) washing the reaction mixture with an aqueous basic solution and at least one additional solvent to obtain a slurry mixture containing precipitated solids, and (ii) filtering the precipitated solids.

In some embodiments, the process comprises (1) preparing the compound having formula (I) by reacting the compound having the formula (II) with dimethylsulphate (DMS), and (2) isolating the compound having formula (I) from the reaction mixture by (i) adding an aqueous basic solution, at least one water immiscible solvent and at least one additional solvent to obtain a slurry mixture containing precipitated solids, and (ii) filtering the precipitated solids.

In some embodiments, the aqueous basic solution comprises DABCO, TBAB, NaOH, K₂CO₃, KHCO₃, Na₂CO₃, Et₃N, NaOMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof. In some embodiments, the aqueous basic solution is an aqueous solution of a base selected from the group consisting of K₂CO₃, KHCO₃, Na₂CO₃, NaHCO₃, K₂CO₃ NH₄OH, NaOH, and any combination thereof. In some embodiments, the aqueous basic solution is an aqueous solution of K₂CO₃.

In some embodiments, the concentration of the base in the aqueous basic solution is 2-18% based on the total weight (w/w). In some embodiments, the concentration of the base in the aqueous basic solution is about 15% based on the total weight (w/w).

In some embodiments, the compound having the formula (I) in the mixture is in salt form.

In some embodiments, the mixture comprises a solvent. In some embodiments, the slurry mixture comprises a solvent.

The mixture comprising the compound having formula (I) may comprise DMS and any or all solvents used during the process for preparing the compound having formula (I).

The additional solvent is a solvent used to isolate or assist in the isolation of the compound having formula (I). The additional solvent may be added directly to the mixture. The additional solvent may also be added with the aqueous basic solution.

In some embodiments, the additional solvent is the same as the solvent(s) used during the process for preparing the compound having formula (I).

In some embodiments, the additional solvent is different from the solvent(s) used during the process for preparing the compound having formula (I).

In some embodiments, the additional solvent is a polar solvent. In some embodiments, the additional solvent is a water immiscible solvent In some embodiments, the polar solvent has a dielectric constant equal to or above 4. In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20. In some embodiments, the polar solvent has a dielectric constant of 4.7.

In some embodiments, the ratio between the additional solvent to the compound having formula (I) is greater than 0.5:1. In some embodiments, the ratio between the additional solvent to the compound having formula (I) is less than 2:1. In some embodiments, the ratio between the additional solvent to the compound having formula (I) is about 1:1.

In some embodiments, the ratio between the additional solvent to the compound having formula (II) used for preparing the compound having formula (I) is greater than 0.5:1. In some embodiments, the ratio between the additional solvent to the compound having formula (II) used for preparing the compound having formula (I) is less than 2:1. In some embodiments, the ratio between the additional solvent to the compound having formula (II) used for preparing the compound having formula (I) is about 1:1.

In some embodiments, the additional solvent is a polar solvent and the polar solvent is CPME.

In some embodiments, the solvent used during the process for preparing the compound having formula (I) is DMA and the additional solvent is CPME.

In some embodiments, the ratio between CPME to the compound having formula (I) is greater than 0.5:1. In some embodiments, the ratio between CPME to the compound having formula (I) is less than 2:1. In some embodiments, the ratio between CPME to the compound having formula (I) is about 1:1.

In some embodiments, the ratio between CPME to the compound having formula (II) used for preparing the compound having formula (I) is greater than 0.5:1. In some embodiments, the ratio between CPME to the compound having formula (II) used for preparing the compound having formula (I) is less than 2:1. In some embodiments, the ratio between CPME to the compound having formula (II) used for preparing the compound having formula (I) is about 1:1.

In some embodiments, the slurry mixture is mixed for 30 minutes to 8 hours.

In some embodiments, the slurry mixture is mixed at temperature between 25 to 60 degrees Celsius. In some embodiments, the slurry mixture is mixed at temperature between 25 to 50 degrees Celsius. In some embodiments, the slurry mixture is mixed at temperature between 25 to 35 degrees Celsius. In some embodiments, the slurry mixture is mixed at temperature of about 30 degrees Celsius.

In some embodiments, the slurry mixture is mixed using mechanical stirrer.

In some embodiments, the slurry mixture is mixed using high shear stirrer.

In some embodiments, the slurry mixture is mixed using both mechanical stirrer and high shear stirrer.

In some embodiments, the slurry mixture is obtained by adding the mixture comprising the compound having formula (I) and DMS and optionally organic solvent into a 2-18% of aqueous basic solution.

In some embodiments, the slurry mixture is obtained by adding the mixture comprising the compound having formula (I) and DMS and optionally organic solvent into a 11-18% of aqueous basic solution.

In some embodiments, the slurry mixture is obtained by adding the mixture comprising the compound having formula (I) and DMS and optionally organic solvent into a 15% of aqueous basic solution.

In some embodiments, the slurry mixture obtained by adding the 2-18% aqueous basic solution into a mixture comprising the compound having formula (I), DMS and optionally an organic solvent.

In some embodiments, in step (2), the precipitated solids are filtered at a temperature between 15 to 45 degrees Celsius. In some embodiments, in step (2), the precipitated solids are filtered at a temperature between 15 to 20 degrees Celsius. In some embodiments, in step (2), the precipitated solids are filtered at a temperature between 20 to 25 degrees Celsius. In some embodiments, in step (2), the precipitated solids are filtered at a temperature between 25 to 30 degrees Celsius. In some embodiments, in step (2), the precipitated solids are filtered at a temperature between 30 to 35 degrees Celsius. In some embodiments, in step (2), the precipitated solids are filtered at a temperature between 35 to 40 degrees Celsius. In some embodiments, in step (2), the precipitated solids are filtered at a temperature between 40 to 45 degrees Celsius.

In some embodiments, the filtered solid obtained in step (2) is washed with an organic solvent during filtration in step (2). In some embodiments, the organic solvent is CPME.

In some embodiments, the filtered solid obtained in step (2) is washed with water during filtration in step (2).

In some embodiments, the filtered solid is mixed with water and stirred for 1 to 3 hours and filtered.

In some embodiments, the filtered solid is mixed with water and stirred at a temperature of 25-50 degrees Celsius and filtered.

In some embodiments, the organic solvent is the same organic solvent as used in obtaining the compound having formula (I).

In some embodiments, the aqueous basic solution is 15% of K₂CO₃ in water based on the total weight (w/w) of K₂CO₃ in water.

In some embodiments, the organic phase is the solution which is obtained in the reaction of compound having the formula (II) with DMS.

In some embodiments, the organic phase is obtained by adding an organic water immiscible solvent to the mixture of the compound having formula (I) and DMS obtained in the reaction of compound (II) and DMS.

In some embodiments, the step of adding an aqueous basic solution comprises additional use of a phase transfer catalyst (PTC) such as tetra-n-butylammonium bromide (TBAB).

In some embodiments, a solution of the compound having formula (I) in CPME is obtained by mixing CPME and the compound having formula (I) in weight ratio of 10:1 prior to washing with 2-18% w/w of aqueous basic solution.

In some embodiments, the solution of the compound having formula (I) in CPME is obtained by warming the combination of CPME and the compound having formula (I) up to 65° C. prior to washing with 2-18% w/w of aqueous basic solution.

In some embodiments, the solution of the compound having formula (I) in CPME is obtained by warming the combination of CPME and the compound having formula (I) up to about 50° C. prior to washing with 2-18% w/w of aqueous basic solution. In some embodiments, the resultant mixture obtained from the reaction of the compound having formula (II) with DMS is dissolved in CPME.

In some embodiments, the resultant mixture obtained from the reaction of the compound having formula (II) with DMS is dissolved with CPME and washed with water base solution.

In some embodiment the resultant mixture is a mixture of the compound having formula (I) with the solvent which was used in the reaction of the compound having formula (II) with DMS.

In some embodiments, the conversion of the 5-fluoro-4-imino-1-(toluene-4-sulfonyl)-3, 4-dihydro-1H-pyrimidin-2-one to 5-fluoro-4-imino-3-methyl-1-(toluene-4-sulfonyl)-3,4-dihydro-1H-pyrimidin-2-one is higher than 50%.

In some embodiments, the chemical yield of 5-fluoro-4-imino-3-methyl-1-(toluene-4-sulfonyl)-3, 4-dihydro-1H-pyrimidin-2-one is higher than 50%. The yield of the purified 5-fluoro-4-imino-3-methyl-1-(toluene-4-sulfonyl)-3, 4-dihydro-1H-pyrimidin-2-one is higher than 50%, 60%, 70%, 80%, 90% or 99%.

In some embodiments, the conversion of the 5-fluoro-4-imino-1-(toluene-4-sulfonyl)-3, 4-dihydro-1H-pyrimidin-2-one to 5-fluoro-4-imino-3-methyl-1-(phenyl-4-sulfonyl)-3,4-dihydro-1H-pyrimidin-2-one is higher than 50%.

In some embodiments, the chemical yield of 5-fluoro-4-imino-3-methyl-1-(phenyl-4-sulfonyl)-3, 4-dihydro-1H-pyrimidin-2-one is higher than 50%. The yield of the purified 5-fluoro-4-imino-3-methyl-1-(toluene-4-sulfonyl)-3, 4-dihydro-1H-pyrimidin-2-one is higher than 50%, 60%, 70%, 80%, 90% or 998.

In some embodiments, the mixture comprises the compound having formula (I), at least one water immiscible solvent, and at least one additional solvent, and the process for isolating the compound having formula (I) comprises (i) washing the mixture with water to obtain slurry mixture containing solids, and (ii) filtering the precipitated solids.

In some embodiments, the multi-phase system comprises a liquid and solids. In some embodiments, wherein the multi-phase system comprises a liquid and solids, the process for isolating the compound having formula (I) from the reaction mixture comprises filtering the solids.

For example, the compound having formula (I) may be isolated from the reaction mixture in according with the process described herein as route 1.

In some embodiments, the mixture comprises the compound having formula (I) and DMS, and the process for isolating the compound having formula (I) from the reaction mixture comprises (1) adding at least one water immiscible solvent and an aqueous basic solution to the mixture to form precipitated solids of the compounds having formula (I) and (2) filtering the precipitated solids.

In some embodiments, the compound having formula (I) in the mixture is in salt form.

In some embodiments, the water-immiscible solvent is polar. In some embodiments, the water-immiscible solvent is polar.

In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20.

In some embodiments, the polar solvent is an organic polar solvent.

In some embodiments, the mixture comprises a solvent selected from the group consisting of DMA, CPME, MeTHF, DMA, DMF, DCM, toluene, anisole, and any combination thereof.

In some embodiments, the mixture comprises a solvent selected from the group consisting of DMA, CPME, MeTHF, DMA, DMF, DCM and any combination thereof.

In some embodiments, the solvent is DMA. In some embodiments, the solvent is CPME. In some embodiments, the solvent is MeTHF.

In some embodiments, the process comprises evaporation of the polar solvent prior to filtration. In some embodiments, the process comprises partial evaporation of the polar solvent prior to filtration.

In some embodiments, the process comprises cooling the reaction mixture prior to filtration.

In some embodiments, step (1) is conducted in the presence of at least one water-immiscible solvent.

In some embodiments, the water immiscible solvent is added after step (1) and before addition of the aqueous basic solution to the reaction mixture.

In some embodiments, the water immiscible solvent is added at the same time as addition of the aqueous basic solution to the reaction mixture.

In some embodiments, the water immiscible solvent is added after addition of the aqueous basic solution to the reaction mixture. In some embodiments, the water immiscible solvent is added immediately after addition of the aqueous basic solution to the reaction mixture.

In some embodiments, the water immiscible solvent is an ether-based solvent, an aromatic solvent, or a mixture thereof.

In some embodiments, the water immiscible solvent is CPME, THE, anisole, toluene, or any mixture thereof.

In some embodiments, the water-immiscible solvent is toluene, anisole, or a combination thereof.

In some embodiments, the water immiscible solvent and the aqueous basic solution are added sequentially.

In some embodiments, the water immiscible solvent is added gradually.

In some embodiments, the aqueous basic solution is added gradually.

In some embodiment, step (1) is conducted in the presence of a solvent selected from the group consisting of DMA, CPME, MeTHF, DMA, DMF, DCM, toluene, anisole, and any combination thereof.

In some embodiments, step (1) is conducted in the presence of at least one water-immiscible solvent.

In some embodiments, the water immiscible solvent includes but is not limited to ether-based solvent, aromatic solvent such as CPME, THE, anisole, toluene, and any mixture thereof.

In some embodiments, the water-immiscible solvent is toluene, anisole, or a combination thereof.

In some embodiments, step (1) is conducted in the presence of a solvent in addition to the water immiscible solvent.

In some embodiments, step (1) is conducted in the presence of a solvent and without a water immiscible solvent.

In some embodiments, the solvent is DMA.

In some embodiments, step (1) is conducted in the presence of a mixture of solvents wherein at least one solvent is water immiscible and at least one solvent is water miscible.

In some embodiments, the mixture of solvents is a mixture of a polar water miscible solvent and a non-polar water-immiscible solvent.

In some embodiments, the mixture of solvents is DMA and anisole. In some embodiments, the weight ratio between DMA and anisole is between 100:1 to 1:1. In some embodiments, the weight ratio between DMA and anisole is about 1:1. In some embodiments, the weight ratio between DMA and the compound having formula (II) is from about 15:1 to about 0.5:1. In some embodiments, the weight ratio between anisole and the compound having formula (II) is from about 10:1 to about 1:1.

In some embodiments, the mixture of solvents is a mixture of DMA and toluene. In some embodiments, the weight ratio between DMA and toluene is between 100:1 to 1:1. In some embodiments, the weight ratio between DMA and toluene is about 1:1. In some embodiments, the weight ratio between DMA and the compound having formula (II) is from about 15:1 to about 0.5:1. In some embodiments, the weight ratio between toluene and the compound having formula (II) is from about 10:1 to about 1:1.

In some embodiments, the compound having formula (I) reacts with DMS in the presence of a base having a pKa equal to or less than the pKa of the compound having formula (I).

During the reaction and before water in any form, such as in the form of an aqueous basic solution, is added to the reaction mixture, the compound having formula (I) may be in salt form. In some embodiments, the compound having formula (I) is in salt form. In some embodiments, the compound having the formula (I) is in salt form during the reaction. In some embodiments, the compound having formula (I) is partially in salt form. In some embodiments, the compound having formula (I) is partially in salt form during the reaction. In some embodiments, the salt of the compound having formula (I) is a monomethylsulfate salt of the compound having formula (I).

In some embodiments, the process comprises (1) preparing the compound having formula (I) by reacting the compound having the formula (II) with dimethylsulphate (DMS) in the presence of at least one water immiscible solvent, and (2) isolating the compound having formula (I) from the reaction mixture by (i) washing the reaction mixture with an aqueous basic solution to from an organic phase and a water phase, (ii) separating the organic phase from the water phase, and (iii) crystalizing the compound having the formula (I) and filtering the crystalized solid.

In some embodiments, step (i) comprises washing the reaction mixture with 2-18% w/w of aqueous basic solution to form an organic phase and a water phase.

In some embodiments, the process comprises (1) preparing the compound having formula (I) by reacting the compound having the formula (II) with dimethylsulphate (DMS), (2) adding at least one water immiscible solvent to the reaction mixture, and (3) isolating the compound having formula (I) from the reaction mixture by (i) washing the reaction mixture with an aqueous basic solution to form an organic phase and a water phase, (ii) separating the organic phase from the water phase, and (iii) crystalizing the compound having the formula (I) and filtering the crystallized solid.

In some embodiments, step (i) comprises washing the reaction mixture with 2-18% w/w of aqueous basic solution to form an organic phase and a water phase.

In some embodiments, the process comprises (1) preparing the compound having formula (I) by reacting the compound having the formula (II) with dimethylsulphate (DMS), and (2) isolating the compound having formula (I) from the reaction mixture by (i) adding at least one water immiscible solvent and an aqueous basic solution to the reaction mixture to form an organic phase and a water phase, (ii) separating the organic phase from the water phase, and (iii) crystalizing the compound having the formula (I) and filtering the crystallized solid.

In some embodiments, the aqueous basic solution is a 2-18% w/w aqueous basic solution.

In some embodiments, the compound having the formula (I) is crystalized by concentrating the organic phase, adding an anti-solvent, and/or seeding.

In some embodiments, the process comprises (1) preparing the compound having formula (I) by reacting the compound having the formula (II) with dimethylsulphate (DMS) in the presence of at least one water immiscible solvent and at least one additional solvent, and (2) isolating the compound having formula (I) from the reaction mixture by (i) washing the reaction mixture with an aqueous basic solution to obtain a slurry mixture comprising precipitated solids, and (ii) filtering the precipitated solids.

In some embodiments, step (i) comprises washing the reaction mixture with 2-18% w/w of aqueous basic solution to obtain a slurry mixture comprising precipitated solids.

In some embodiments, the process comprises (1) preparing the compound having formula (I) by reacting the compound having the formula (II) with dimethylsulphate (DMS), (2) adding at least one water immiscible solvent and at least one additional solvent to the reaction mixture, and (3) isolating the compound having formula (I) from the reaction mixture by (i) washing the reaction mixture with an aqueous basic solution to obtain a slurry mixture containing precipitated solids, and (ii) filtering the precipitated solids.

In some embodiments, step (i) comprises washing the reaction mixture with 2-18% w/w of aqueous basic solution to obtain a slurry mixture comprising precipitated solids.

In some embodiments, the process comprises (1) preparing the compound having formula (I) by reacting the compound having the formula (II) with dimethylsulphate (DMS), (2) adding at least one water immiscible solvent to the reaction mixture, and (3) isolating the compound having formula (I) from the reaction mixture by (i) washing the reaction mixture with an aqueous basic solution and at least one additional solvent to obtain a slurry mixture containing precipitated solids, and (ii) filtering the precipitated solids.

In some embodiments, step (i) comprises washing the reaction mixture with 2-18% w/w of aqueous basic solution to obtain a slurry mixture comprising precipitated solids.

In some embodiments, the process comprises (1) preparing the compound having formula (I) by reacting the compound having the formula (II) with dimethylsulphate (DMS), and (2) isolating the compound having formula (I) from the reaction mixture by (i) adding an aqueous basic solution, at least one water immiscible solvent and at least one additional solvent to obtain a slurry mixture containing precipitated solids, and (ii) filtering the precipitated solids.

In some embodiments, the aqueous basic solution is a 2-18% w/w aqueous basic solution.

In some embodiments, the aqueous basic solution is an aqueous solution comprising DABCO, TBAB, NaOH, K₂CO₃, KHCO₃, Na₂CO₃, Et₃N, NaoMe, NaOEt, or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof. In some embodiments, the aqueous basic solution is an aqueous solution of a base selected from the group consisting of K₂CO₃, KHCO₃, Na₂CO₃, NaHCO₃, K₂CO₃ NH₄OH, NaOH, and any combination thereof. In some embodiments, the aqueous basic solution is an aqueous solution of K₂CO₃.

In some embodiments, the concentration of the base in the aqueous basic solution is 2-18% based on the total weight (w/w). In some embodiments, the concentration of the base in the aqueous basic solution is about 15% based on the total weight (w/w).

In some embodiments, the additional solvent is different from the water immiscible solvent.

In some embodiments, the additional solvent is a polar solvent.

In some embodiments, the polar solvent has a dielectric constant equal to or above 5.

In some embodiments, the ratio between the additional solvent to the compound having formula (I) or formula (II) is about 1:1.

In some embodiments, the additional solvent is CPME.

In some embodiments, the slurry mixture is mixed at a temperature between 25 to 50 degrees Celsius.

In some embodiments, in step (2), the precipitated solids are filtered at a temperature between 15 to 45 degrees Celsius.

In some embodiments, the water immiscible solvent is polar.

In some embodiments, the water immiscible solvent is non-polar.

In some embodiments, the reaction of the compound having formula (II) with DMS is conducted in the absence of base.

In some embodiments, the reaction of the compound having formula (II) with DMS is conducted in the presence of at least one base.

In some embodiments, the base is selected from the group consisting of DABCO, TBAB, NaOH, K₂CO₃, KHCO₃, Na₂CO₃, Et₃N, NaoMe, NaOEt and any combination thereof.

In some embodiments, the base is selected from the group consisting of TBAB, NaOH, Na₂CO₃, Et₃N, NaOMe, and any combination thereof.

In some embodiments, when a base is present in the reaction of the compound having formula (II) with DMS, and the base has a pKa that is higher than the pKa of the compound having formula (I), the compound having formula (I) is not in salt form. In some embodiments, the reaction of the compound having formula (II) with DMS is conducted at a temperature between 25-85° C. In some embodiments, the temperature is between 25-50° C. In some embodiments, the reaction of the compound having formula (II) with DMS is conducted at a temperature between 35-50° C.

In some embodiments, the molar ratio between the compound having formula (II) and DMS is between 1:2 to 1:10. In some embodiments, the molar ratio between the compound having formula (II) and DMS is between 1:2 to 1:5. In some embodiments, the molar ratio between the compound having formula (II) and DMS is between 1:2 to 1:4. In some embodiments, the molar ratio between the compound having formula (II) and DMS is about 1:3. In some embodiments, the molar ratio between the compound having formula (II) and DMS is about 1:3.5.

In some embodiments, the molar ratio between the compound having formula (II) and the base is 1:0.1 to 1:10. In some embodiments, the molar ratio between the compound having formula (II) and the base is 1:0.1 to 1:5.5.

In some embodiments, the base is added after 4 hours from the beginning of the reaction of compound (II) with DMS. Suitable bases include alkoxides and carbonates.

In some embodiments, the base is added after 4 hours from the beginning of the reaction of the compound having formula (II) with DMS and the temperature of the reaction is above 30 degrees Celsius.

In some embodiments, the base is added at the beginning of the reaction. Suitable bases include DABCO, NEt₃, LiCO₃, and KHCO₃.

When the base is added at the beginning of the reaction of the compound having formula (II) with DMS, the temperature is preferably above 30 degrees Celsius.

In some embodiments, the reaction of the compound having formula (II) with DMS is conducted in the presence of at least one solvent.

In some embodiments, the reaction of the compound having formula (II) with DMS is conducted in the presence of two solvents.

In some embodiments, the reaction of the compound having formula (II) with DMS is conducted in the presence of one solvent and the solvent is water immiscible.

In some embodiments, the reaction of the compound having formula (II) with DMS is conducted in the presence of two or more solvents and at least one of the solvents is water immiscible. The other solvent(s) may be water immiscible or water miscible.

In some embodiments, the solvent is a polar solvent. In some embodiments, the solvent is a non-polar solvent.

In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20.

In some embodiments, the solvent is selected from the group consisting of DMA, CPME, MeTHF, DMA, DMF, DCM, toluene, anisole and any combination thereof.

In some embodiments, the solvent is selected from the group consisting of DMA, CPME, MeTHF, DMA, DMF, DCM and any combination thereof.

In some embodiments, the solvent is DMA. In some embodiments, the solvent is CPME. In some embodiments, the solvent is MeTHF. In some embodiments, the solvent is toluene. In some embodiments, the solvent is anisole.

In some embodiments, the solvent(s) is a solvent that dissolves the compound of formula (I) completely.

In some embodiments, the solvent is MeTHF and the base is NEt₃.

In some embodiments, the solvent is a mixture of at least two solvents.

In some embodiments, the solvent is a mixture of DMA and CPME.

In some embodiments, the weight ratio between DMA and CPME is between 1:1 to 1:10. In some embodiments, the weight ratio between DMA and CPME is between 1:1 to 1:4. In some embodiments, the weight ratio between DMA and CPME is between 1:2 to 1:4. In some embodiments, the weight ratio between DMA and CPME is between 1:3 to 1:5. In some embodiments, the weight ratio between DMA and CPME is about 1:4.

In some embodiments, the solvent is a mixture of DMA and MeTHF.

In some embodiments, the solvent is a mixture of DMA and MeTHF in a weight ratio of 1:1 to 1:4. In some embodiments, the solvent is a mixture of DMA and MeTHF in a weight ratio of 1:2 to 1:4.

In some embodiments, the alkylation process is conducted in the presence of a mixture of DMA and CPME in a weight ratio of 1:2 to 1:4. In some embodiments, the alkylation process is conducted in the presence of a mixture of DMA and CPME in a weight ratio of 1:4.

In some embodiments, the solvent is a mixture of a polar water-miscible solvent and a non-polar water-immiscible solvent.

In some embodiments, the solvent is a mixture of DMA and anisole.

In some embodiments, the weight ratio between DMA and anisole is between 100:1 to 1:1. In some embodiments, the weight ratio between DMA and anisole is between 75:1 to 1:1. In some embodiments, the weight ratio between DMA and anisole is between 50:1 to 1:1. In some embodiments, the weight ratio between DMA and anisole is between 25:1 to 1:1. In some embodiments, the weight ratio between DMA and anisole is about 1:1.

In some embodiments, the solvent is a mixture of DMA and toluene.

In some embodiments, the weight ratio between DMA and toluene is between 100:1 to 1:1. In some embodiments, the weight ratio between DMA and toluene is between 75:1 to 1:1. In some embodiments, the weight ratio between DMA and toluene is between 50:1 to 1:1. In some embodiments, the weight ratio between DMA and toluene is between 25:1 to 1:1. In some embodiments, the weight ratio between DMA and toluene is about 1:1.

In some embodiments, in the reaction of the compound having formula (II) with DMS, the molar ratio between the solvent or mixture of solvents to the compound having formula (II) is between 30:1 and 1:1. In some embodiments, in the reaction of the compound having formula (II) with DMS, the molar ratio between the solvent or mixture of solvents to the compound having formula (II) is between 20:1 and 5:1. In some embodiments, in the reaction of the compound having formula (II) with DMS, the molar ratio between the solvent or mixture of solvents to the compound having formula (II) is between 15:1 and 10:1. In some embodiments, in the reaction of the compound having formula (II) with DMS, the molar ratio between the solvent or mixture of solvents to the compound having formula (II) is between 14:1 to 12:1. In some embodiments, in the reaction of the compound having formula (II) with DMS, the molar ratio between the solvent or mixture of solvents to the compound having formula (II) is about 13:1. In some embodiments, in the reaction of the compound having formula (II) with DMS, the molar ratio between the solvent or mixture of solvents to the compound having formula (II) is 12.7:1.

In some embodiments, in the reaction of the compound having formula (II) with DMS, the molar ratio between the solvent or mixture of solvents to DMS is between 10:1 to 1:1. In some embodiments, in the reaction of the compound having formula (II) with DMS, the molar ratio between the solvent or mixture of solvents to DMS is between 5:1 to 3:1. In some embodiments, in the reaction of the compound having formula (II) with DMS, the molar ratio between the solvent or mixture of solvents to DMS is about 4:1. In some embodiments, in the reaction of the compound having formula (II) with DMS, the molar ratio between the solvent or mixture of solvents to DMS is 3.9:1.

In some embodiments, the reaction of the compound having formula (II) with DMS further comprises neutralizing with an aqueous basic solution.

In some embodiments, the excess DMS is neutralized with an aqueous basic solution.

In some embodiments, the weight ratio of the DMA:CPME:compound having the formula (II) is 1.5:0:1 to 1:5:1.

In some embodiments, the weight ratio between DMA and the compound having formula (II) is from about 15:1 to about 0.5:1. In some embodiments, the weight ratio between anisole and the compound having formula (II) is from about 10:1 to about 1:1.

In some embodiments, the weight ratio between DMA and the compound having formula (II) is from about 15:1 to about 0.5:1. In some embodiments, the weight ratio between toluene and the compound having formula (II) is from about 10:1 to about 1:1.

In some embodiments, the base in the aqueous basic solution may include but is not limited to K₂CO₃, KHCO₃, Na₂CO₃, NaHCO₃, K₂CO₃ NH₄OH, NaOH or any combination thereof. In some embodiments, the concentration of the base in the aqueous basic solution is 2-18% based on the total weight (w/w).

In some embodiments, the base in the aqueous basic solution is K₂CO₃.

In some embodiments, the step of adding an aqueous basic solution comprises additional use of a phase transfer catalyst (PTC) such as tetra-n-butylammonium bromide (TBAB).

In some embodiments, the reaction of the compound having formula (II) with DMS to obtain the compound having formula (I) has a yield of at least 60%. In some embodiments, the reaction of the compound having formula (II) with DMS to obtain the compound having formula (I) has a yield of at least 70%. In some embodiments, the reaction of the compound having formula (II) with DMS to obtain the compound having formula (I) has a yield of at least 80%.

The compound having formula (II) may be prepared using any process known in the art including, but not limited to, the process described in PCT International Application Publication Nos. WO 2015/103142 and WO 2015/103144, the entire content of each of which is hereby incorporated by reference.

In some embodiments, the compound having formula (IIai) is prepared by contacting a compound having formula (IV):

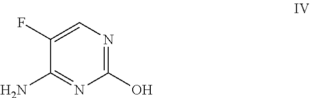

with bis-N, O-trimethylsilylacetamide (BSA) and forming a compound having formula (IIai),
wherein the molar ratio of the compound having formula (IV) to bis-N, O-trimethylsilylacetamide (BSA) is 1:1.1 and the contacting step is carried out at a temperature from about 22° C. to about 70° C.

In some embodiments, the contacting step further includes contacting the compound having formula (IV) with CH₃CN.

In some embodiment, the process comprises contacting a BSA treated reaction mixture with an arylsulfonyl chloride.

In some embodiments, the molar ratio between the compound having formula (IV) to arylsulfonyl chloride is from about 1:2 to about 2:1. In some embodiments, the molar ratio between the compound having formula (IV) to arylsulfonyl chloride 1:1.1.

In some embodiments, the compound having formula (IIai) may be prepared by contacting a compound having formula (IV) with bis-N, O-trimethylsilylacetamide (BSA) at an elevated temperature, such as 70° C., for a period of about 1 hour (h), followed by cooling and contacting the solution containing the protected pyrimidinol with CH₃-PhSO₂Cl at about 20° C.-25° C. In some embodiments, the molar ratio between the compound having formula (IV) to BSA and the sulfonyl chloride is about 1:3:1.1, respectively. In some embodiments, reducing the molar ratio of the reactants to about 1:1.1:1.1 affords improved yields.

The compound having formula (II) may be prepared using the process described in PCT International Application Publication No. WO/2021/181274, the entire content of which is hereby incorporated by reference.

In some embodiments, the compound having formula (II) is prepared by reacting 5-fluorocytosine with compound having the formula (III):

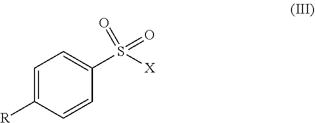

in the presence of at least one polar solvent and at least one base, wherein:
   R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃; and
      X is a halogen or —OSO₂PhR.

In some embodiments, the compound having the formula (II) is (IIa) wherein R is alkyl.

In some embodiments, the compound having the formula (IIa) is (IIai) wherein R is methyl.

In some embodiments, the compound having formula (II) is a compound having formula (IIai)

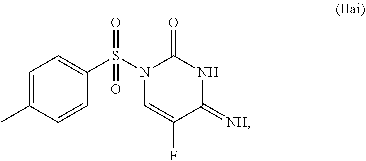 [SMILES: Cc1ccc(S(=O)(=O)n2cc(F)c(=N)[nH]c2=O)cc1]

and the compound having formula (IIai) may be prepared by reacting 5-fluorocytosine with compound having the formula (III):

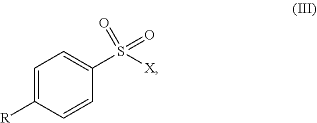

wherein R is methyl and X is a halogen or —OSO₂PhR, in the presence of at least one polar solvent and at least one base to obtain the compound of formula (IIai).

The present invention also provides a process for obtaining the compound 5-fluoro-4-imino-3-methyl-1-(phenyl-4-sulfonyl)-3, 4-dihydro-1H-pyrimidin-2-one having formula (I):

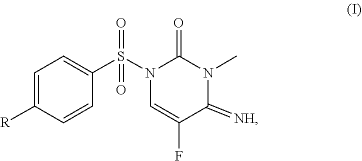

comprising:
(a) reacting 5-fluorocytosine with compound having formula (III):

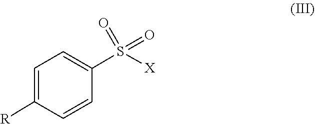

in the presence of at least one polar solvent and at least one base to obtain the compound having formula (II)

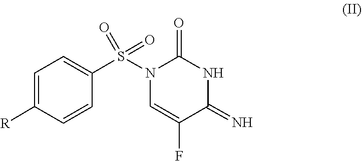

and
(b) preparing the compound having formula (I) and isolating the compound having formula (I) from the reaction mixture in accordance with any one of the processes described herein.

In some embodiments, R is alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃.

In some embodiments, the compound having the formula (I) is (Ia) wherein R is alkyl.

In some embodiments, the compound having the formula (Ia) is (Iai) wherein R is methyl.

In some embodiments, the compound having formula (I) is a compound having formula (Iai):

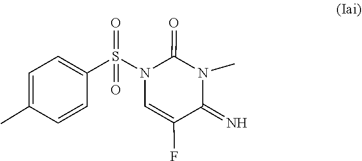

and the process comprises:
(a) reacting 5-fluorocytosine with a compound having formula (III):

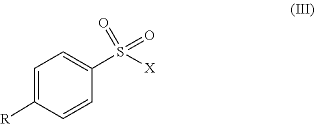

in the presence of at least one polar solvent and at least one base to obtain a compound having formula (IIai)

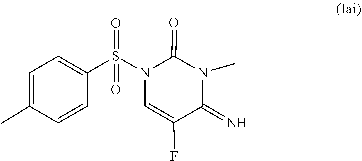

and
(b) preparing the compound having formula (I) and isolating the compound having formula (I) from the reaction mixture in accordance with any one of the processes described herein,

wherein R is methyl and X is a halogen or —OSO₂PhR.

During the reaction and before water in any form is added to the reaction mixture the compound having formula (I) may be in salt form. Water may be added in the form of an aqueous basic solution. In some embodiments, the compound having formula (I) is in salt form. In some embodiments, the compound having the formula (I) is in salt form during the reaction. In some embodiments, the compound having formula (I) is partially in salt form. In some embodiments, the compound having formula (I) is partially in salt form during the reaction. In some embodiments, the salt of the compound having formula (I) is a monomethylsulfate salt of the compound having formula (I).

In some embodiments, a salt or salt mixture is optionally added to a reaction mixture after DMS is added to the reaction. The salt or the salt mixture is prepared in a previous alkylation reaction.

In some embodiments, the compound having the formula (I) obtained in the alkylation is suspended in the solvent. In some embodiments, the compound having the formula (I) obtained in the alkylation is soluble in the solvent. In some embodiments, the salt of the compound having the formula (I) is suspended in the solvent. In some embodiments, the salt of the compound having the formula (I) is soluble in the solvent. In some embodiments, the salt or salt solution is added to alkylation reaction after the addition of dimethylsulfate.

The present invention provides a monomethylsulfate salt of the compound having formula (I).

In some embodiments, the reaction of 5-fluorocytosine with the compound having formula (III) is carried out in the absence of protecting group.

In some embodiments, X is a halogen. In some embodiments, the halogen is Cl, Br or I. In some embodiments, the halogen is Cl.

In some embodiments, X is —SO₂PhR, wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃.

In some embodiments, X is

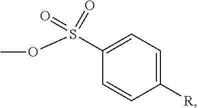

wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃.

In some embodiments, the compound having formula (III) is a compound having formula (IIIb)

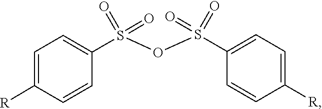

wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃. In some embodiments, X is —SO₂PhR and R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃.

In some embodiments, R is alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃.

In some embodiments, R is alkyl.

In some embodiments, the compound having formula (III) is toluenesulfonyl anhydride.

In some embodiments, the compound having the formula (III) may include but is not limited to 4-toluenesulfonyl chloride (TsCl) and toluenesulfonyl anhydride.

In some embodiments, the compound having the formula (III) is 4-toluenesulfonyl chloride (TsCl). In some embodiments, the compound having the formula (III) is toluenesulfonyl anhydride.

In some embodiments, the reaction of 5-fluorocytosine with the compound having the formula (III) is conducted at a temperature between (−5)-85° C. In some embodiments, the reaction of 5-fluorocytosine with the compound having the formula (III) is conducted at temperature between (−5)-25° C. In some embodiments, the reaction of 5-fluorocytosine with the compound having the formula (III) is conducted at temperature between (−5)-5° C. In some embodiments, the reaction of 5-fluorocytosine with the compound having the formula (III) is conducted at temperature between (−5)-0° C. In some embodiments, the reaction of 5-fluorocytosine with the compound having the formula (III) is conducted at temperature between 0-5° C. In some embodiments, the reaction of 5-fluorocytosine with the compound having the formula (III) is conducted at temperature between 5-25° C. In some embodiments, the reaction of 5-fluorocytosine with the compound having the formula (III) is conducted at temperature between 25-85° C. In some embodiments, the reaction of 5-fluorocytosine with the compound having the formula (III) is conducted in the presence of at least one polar solvent and at least one base.

In some embodiments, the reaction of 5-fluorocytosine with the compound having the formula (III) is conducted in the presence of at least one polar solvent, at least one base and at a temperature between 0-5° C. In some embodiments, the reaction of 5-fluorocytosine with the compound having the formula (III) is conducted in the presence of two polar solvents, one base and at a temperature between 0-5° C.

In some embodiments, the reaction of 5-fluorocytosine with the compound having the formula (III) is conducted in the presence of at least one polar solvent, at least one base and at a temperature between 5-25° C.

In some embodiments, the reaction of 5-fluorocytosine with the compound having the formula (III) is conducted in the presence of a polar solvent, at least one base and at a temperature between 25-85° C.

In some embodiments, the reaction of 5-fluorocytosine with compound having the formula (III) is conducted in the presence of at least one polar solvent, at least one base and at a temperature between (−5)-85° C.

In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20.

In some embodiments, wherein R is methyl in the compound having formula (III), the sulfonation step is a tosylation step.

In the tosylation step, the polar solvent has a dielectric constant equal to or above 20.

In some embodiments, the polar solvent having dielectric constant equal to or above 20 may include but is not limited to dimethyl acetamide (DMA), N-methylpyrolidone (NMP), acetonitrile (ACN or MeCN), dimethylsulfoxide (DMSO), dimethylformamide (DMF), water or any combination thereof.

In some embodiments, the polar solvent is selected from the group consisting of dimethyl acetamide (DMA), N-methylpyrolidone (NMP), acetonitrile (ACN MeCN), or dimethylsulfoxide (DMSO), dimethylformamide (DMF), dimethylbenzylamine (DMBA), water and any combination thereof.

In some embodiments, the polar solvent is selected from the group consisting of dimethyl acetamide (DMA), acetonitrile (ACN or MeCN), dimethylbenzylamine (DMBA), water and any combination thereof.

In some embodiments the combination of polar solvent and base consist of one phase system.

In some embodiments, the reaction of 5-fluorocytosine with the compound having the formula (III) is conducted in the presence of one polar solvent.

In some embodiments, the reaction of 5-fluorocytosine with the compound having the formula (III) is conducted in the presence of two polar solvents.

In some embodiments, the two polar solvents are selected from the group consisting of dimethyl acetamide (DMA), N-methylpyrolidone (NMP), acetonitrile (ACN or MeCN), dimethylsulfoxide (DMSO), dimethylformamide (DMF), dimethylbenzylamine (DMBA), and water.

In some embodiments, the two polar solvent are selected from the group consisting of dimethyl acetamide (DMA), acetonitrile (ACN or MeCN), dimethyl aminopyridine (DMAP), and water.

In some embodiments, the two polar solvents are DMA and water.

In some embodiments, the weight ratio between the two polar solvents is between 10:1 to 1:10.

In some embodiments, the weight ratio between the two polar solvents is between 2:1 to 1:2.

In some embodiments, the weight ratio between the two polar solvents is 1:1.

In some embodiments, the reaction of 5-fluorocytosine with the compound having the formula (III) is conducted in the presence of three polar solvents.

In some embodiments, the three polar solvents are DMA, water and DMBA.

In some embodiments, at least one base is an organic base.

In some embodiments, at least one base is an inorganic base.

In some embodiments, the reaction of 5-fluorocytosine with the compound having the formula (III) is conducted in the presence of one base.

In some embodiments, the reaction of 5-fluorocytosine with the compound having the formula (III) is conducted in the presence of two bases.

In some embodiments, the base may include but is not limited to K₂CO₃, Na₂CO₃, Li₂CO₃, NaHCO₃, KHCO₃, NaOH, KOH, Et₃N, dimethyl aminopyridine (DMAP), dimethylbenzylamine (DMBA) or any combination thereof.

In some embodiments, the base is selected from a group consisting of K₂CO₃, Na₂CO₃, Li₂CO₃, NaHCO₃, KHCO₃, Et₃N, dimethyl aminopyridine (DMAP) and any combination thereof.

In some embodiments, the base is selected from the group consisting of K₂CO₃, Na₂CO₃, NaOH, KOH, Et₃N, dimethyl aminopyridine (DMAP), and any combination thereof.

In some embodiments, the base is K₂CO₃. In some embodiments, the base is Na₂CO₃. In some embodiments, the base is NaOH. In some embodiments, the base is KOH. In some embodiments, the base is Et₃N. In some embodiments, the base is DMAP.

In some embodiments, the reaction of 5-fluorocytosine with the compound having the formula (III) is conducted in the presence of a DMA and at least one base.

In some embodiments, the reaction of 5-fluorocytosine with the compound having the formula (III) is conducted in the presence of a DMA, water and at least one base. In some embodiments, the reaction of 5-fluorocytosine with the compound having the formula (III) is conducted in the presence of a DMA, water and one base.

In some embodiments, the reaction of 5-fluorocytosine with the compound having the formula (III) is conducted in the presence of a DMA, water and two bases.

In some embodiments, the reaction of 5-fluorocytosine with the compound having the formula (III) is conducted in the presence of a DMA and two bases.

In some embodiments, the two bases are Et₃N and DMAP.

In some embodiments, the two bases are NaOH and DMAP.

In some embodiments, the two bases are dimethylbenzylamine and NaOH.

In some embodiments, the two bases are DMAP and Na₂CO₃.

In some embodiments, the two bases are DMAP and KOH.

In some embodiments, the reaction of 5-fluorocytosine with the compound having the formula (III) is conducted in the presence of DMA, water and K₂CO₃.

In some embodiments, the reaction of 5-fluorocytosine with the compound having the formula (III) is conducted in the presence of DMA and Et₃N.

In some embodiments, the reaction of 5-fluorocytosine with the compound having the formula (III) is conducted in the presence of acetonitrile (ACN or MeCN) and triethylamine (Et₃N).

In some embodiments, the reaction of 5-fluorocytosine with the compound having the formula (III) is conducted in the presence of DMA, water and DMAP.

In some embodiments, the reaction of 5-fluorocytosine with the compound having the formula (III) is conducted in the presence of DMA and Na₂CO₃.

In some embodiments, the reaction of 5-fluorocytosine with the compound having the formula (III) is conducted in the presence of DMBA and Na₂CO₃.

In some embodiments, the reaction of 5-fluorocytosine with the compound having the formula (III) is conducted in the presence of water and DMAP.

In some embodiments, the reaction of 5-fluorocytosine with the compound having the formula (III) is conducted in the presence of DMBA, DMA, water and KOH.

In some embodiments, the reaction of 5-fluorocytosine with the compound having the formula (III) is conducted in the presence of DMBA, DMA, water and NaOH.

In some embodiments, the reaction of 5-fluorocytosine with the compound having the formula (III) is conducted in the presence of a DMA, Et₃N and DMAP.

In some embodiments, the reaction of 5-fluorocytosine with the compound having the formula (III) is conducted in the presence of a DMA, water, NaOH and DMAP.

In some embodiments, the reaction of 5-fluorocytosine with the compound having the formula (III) is conducted in the presence of DMAP and Na₂CO₃.

In some embodiments, the reaction of 5-fluorocytosine with the compound having the formula (III) is conducted in the presence of DMBA and KOH.

In some embodiments, the reaction of 5-fluorocytosine with the compound having the formula (III) is conducted in the presence of DMBA and NaOH.

In some embodiments, the polar solvent has a dielectric constant equal to or above 20 is DMA, and the base is Et₃N.

In some embodiment, the polar solvent has a dielectric constant equal to or above 20 is mixture of DMA and water, and the base is K₂CO₃.

In some embodiments, the polar solvent has a dielectric constant equal to or above 20 is water, the base is Et₃N and the temperature is (−5)-5° C.

In some embodiments, the temperature is (−5)-5° C., and the solvent is acetonitrile.

In another preferred embodiment, the temperature is (−5)-5° C., the solvent is acetonitrile, and the base is Et₃N.

In some embodiments, in the reaction of 5-fluorocytosine with the compound having formula (III), the molar ratio between 5-fluorocytosine and the compound having formula (III) is between 1:10 to 10:1. In some embodiments, in the reaction of 5-fluorocytosine with the compound having formula (III), the molar ratio between 5-fluorocytosine and the compound having formula (III) is between 1:5 to 5:1. In some embodiments, in the reaction of 5-fluorocytosine with the compound having formula (III), the molar ratio between 5-fluorocytosine and the compound having formula (III) is between 1:2 to 2:1. In some embodiments, in the reaction of 5-fluorocytosine with the compound having formula (III), the molar ratio between 5-fluorocytosine and the compound having formula (III) is between 1:1 to 1:2. In some embodiments, in the reaction of 5-fluorocytosine with the compound having formula (III), the molar ratio between 5-fluorocytosine and the compound having formula (III) is about 1:1. In some embodiments, in the reaction of 5-fluorocytosine with the compound having formula (III), the molar ratio between 5-fluorocytosine and the compound having formula (III) is about 1:1.1. In some embodiments, in the reaction of 5-fluorocytosine with the compound having formula (III), the molar ratio between 5-fluorocytosine and the compound having formula (III) is 1:1.2.

In some embodiments, in the reaction of 5-fluorocytosine with the compound having formula (III), the molar ratio between 5-fluorocytosine and the base is between 1:10 to 10:1. In some embodiments, in the reaction of 5-fluorocytosine with the compound having formula (III), the molar ratio between 5-fluorocytosine and the base is between 1:5 to 5:1. In some embodiments, in the reaction of 5-fluorocytosine with the compound having formula (III), the molar ratio between 5-fluorocytosine and the base is between 1:2 to 2:1. In some embodiments, in the reaction of 5-fluorocytosine with the compound having formula (III), the molar ratio between 5-fluorocytosine and the base is between 1:1 to 1:2. In some embodiments, in the reaction of 5-fluorocytosine with the compound having formula (III), the molar ratio between 5-fluorocytosine and the base is about 1:1. In some embodiments, in the reaction of 5-fluorocytosine with the compound having formula (III), the molar ratio between 5-fluorocytosine and the base is 1:1.2. In some embodiments, in the reaction of 5-fluorocytosine with the compound having formula (III), the molar ratio between 5-fluorocytosine and the base is 1:1.3.

In some embodiments, in the reaction of 5-fluorocytosine with the compound having formula (III), the molar ratio between the compound having formula (III) and the base is between 1:10 to 10:1. In some embodiments, in the reaction of 5-fluorocytosine with the compound having formula (III), the molar ratio between the compound having formula (III) and the base is between 1:5 to 5:1. In some embodiments, in the reaction of 5-fluorocytosine with the compound having formula (III), the molar ratio between the compound having formula (III) and the base is between 1:2 to 2:1. In some embodiments, in the reaction of 5-fluorocytosine with the compound having formula (III), the molar ratio between the compound having formula (III) and the base is between 1:1 to 1:2. In some embodiments, in the reaction of 5-fluorocytosine with the compound having formula (III), the molar ratio between the compound having formula (III) and the base is about 1:1. In some embodiments, in the reaction of 5-fluorocytosine with the compound having formula (III), the molar ratio between the compound having formula (III) and the base is 1:1.2.

In some embodiments, the reaction of 5-fluorocytosine with the compound having formula (III) to obtain the compound having formula (II) has a yield of at least 61%.

In some embodiments, the reaction of 5-fluorocytosine with the compound having formula (III) to obtain the compound having formula (II) has a yield higher than 60%, 70%, 80%, 90% or 99%. In some embodiments, the reaction of 5-fluorocytosine with the compound having formula (III) to obtain the compound having formula (II) has a yield higher than 90%.

In some embodiments, the impurities (A) and (B) in the invention process disclosed herein to obtain compound having the formula (II) is less than 20% based on the conversion. In some embodiments, the impurities (A) and (B) in the invention process disclosed herein to obtain compound having the formula (II) is less than 10% based on the conversion. In some embodiments, the impurities (A) and (B) in the invention process disclosed herein to obtain compound having the formula (II) is less than 5% based on the conversion. In some embodiments, the impurities (A) and (B) in the invention process disclosed herein to obtain compound having the formula (II) is less than 3% based on the conversion.

In some embodiments, the reaction of 5-fluorocytosine with the compound having formula (III) to obtain compound having the formula (II) further comprises a step of isolating the compound having formula (II) from the reaction mixture.

In some embodiments, isolation of the compound having formula (II) comprises (i) adding a protic solvent to the reaction mixture to precipitate the compound having formula (II) from the reaction mixture, and (ii) collecting the precipitated compound of formula (II).

In some embodiments, the protic solvent is water, methanol or a combination thereof.

The present invention also provides a compound having formula (II) prepared using the process described herein.

The present invention also provides a compound having formula (IIai) prepared using the process described herein.

In some embodiments, the compound having formula (I) is a compound having formula (Iai):

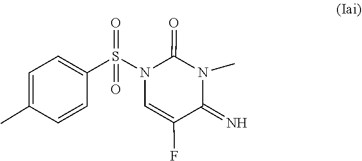

and the compound having formula (II) is a compound having formula (IIai)

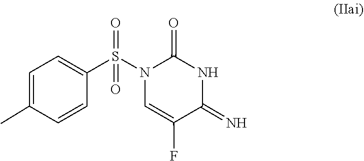

wherein R is methyl and X is a halogen or —OSO₂PhR.

The present invention also provides a compound having formula (I) obtained using any one of the processes described herein.

The present invention also provides a compound having formula (Iai) obtained using any one of the processes described herein.

The present invention also provides a method for isolating a compound having formula (I) from a mixture comprising the compound having formula (I), wherein the method comprises (i) preparing a multi-phase system comprising the compound having formula (I), a water-immiscible solvent and water, and (ii) obtaining and isolating solids of the compound having formula (I) from the multi-phase system.

In some embodiments, the water is added in the form of an aqueous basic solution.

In some embodiments, the aqueous basic solution comprises DABCO, TBAB, NaOH, K₂CO₃, KHCO₃, Na₂CO₃, Et₃N, NaoMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof. In some embodiments, the aqueous basic solution is an aqueous solution of a base selected from the group consisting of K₂CO₃, KHCO₃, Na₂CO₃, NaHCO₃, K₂CO₃ NH₄OH, NaOH, and any combination thereof. In some embodiments, the aqueous basic solution is an aqueous solution of K₂CO₃.

In some embodiments, the concentration of the base in the aqueous basic solution is 2-18% based on the total weight (w/w). In some embodiments, the concentration of the base in the aqueous basic solution is about 15% based on the total weight (w/w).

In some embodiments, the multi-phase system is a mixture comprising a liquid and solids, wherein the method comprises filtering the solids. Preferred embodiments are described in route 1 below.

In some embodiments, the multi-phase system comprises an organic phase and a water phase, and the method comprises separating the organic phase from the water phase, crystallizing the compound having formula (I) from the organic phase, and filtering the crystals. Preferred embodiments are described in route 2 below.

In some embodiments, the multi-phase system is a slurry mixture comprising solids, and the method comprises filtering the solids. Preferred embodiments are described in route 3 below.

In some embodiments, the compound having formula (I) is a compound having formula (Iai):

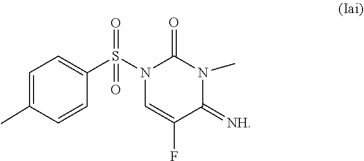

Methods for isolating a compound having formula (I) from a mixture thereof, including routes 1-3, are described below.

The methods for isolating a compound having formula (I) may be applied to any mixture comprising the compound having formula (I), including the reaction mixtures of the processes described herein for preparing the compound having formula (I) and the reaction mixtures of the processes described in PCT International Application Publication Nos. WO 2015/103142, WO 2015/103144, and WO/2021/181274 for preparing the compound having formula (I).

The mixture, including reaction mixture resulting from the alkylation step of the process for preparing the compound having formula (I), may comprise a non-salt form of the compound having the formula (I), a salt form of the compound having the formula (I), or a mixture thereof.

In some embodiments, the step after reacting compound having formula (II) with DMS to give the salt and/or non-salt form of the compound having the formula (I) is defined as the isolation step. In some embodiments, a salt form of the compound having formula (I) is formed prior to the isolation step. In some embodiments, a non-salt form of the compound having formula (I) is formed prior to the isolation step. In some embodiments, the non-salt form of compound having formula (I) is obtained by adding water after reaction with DMS with compound having formula (II). In some embodiments, isolation includes neutralization of the salt form of the compound having the formula (I).

### Isolation of Compound Having the Formula (I), Route 1

The present invention also provides a method for isolating a compound having the formula (I) from a mixture comprising the compound having formula (I) and DMS, wherein the method comprises (1) adding at least one water immiscible solvent and an aqueous basic solution to the mixture to form precipitated solids of the compounds having formula (I) and (2) filtering the precipitated solids.

In some embodiments, the compound having formula (I) is a compound having formula (Iai):

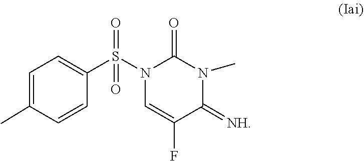

In some embodiments, the compound having formula (I) in the mixture is in salt form.

In some embodiments, the water-immiscible solvent is polar. In some embodiments, the water-immiscible solvent is polar.

In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20.

In some embodiments, the polar solvent is an organic polar solvent.

In some embodiments, the mixture comprises a solvent selected from the group consisting of DMA, CPME, MeTHF, DMA, DMF, DCM, toluene, anisole, and any combination thereof.

In some embodiments, the mixture comprises a solvent selected from the group consisting of DMA, CPME, MeTHF, DMA, DMF, DCM and any combination thereof.

In some embodiments, the solvent is DMA. In some embodiments, the solvent is CPME. In some embodiments, the solvent is MeTHF.

In some embodiments, the method comprises evaporation of the polar solvent prior to filtration. In some embodiments, the method comprises partial evaporation of the polar solvent prior to filtration.

In some embodiments, the method comprises cooling the reaction mixture prior to filtration.

The method for isolating a compound having formula (I) from a mixture thereof may be used to isolate a compound having formula (I) from any mixture thereof, including, but not limited to, (i) the reaction mixture after preparing the compound having formula (I) using the processes described herein, (ii) the reaction mixture after preparing the compound having formula (I) using the processes described in PCT International Application Publication Nos. WO2015/103144 and WO2015/103142, and (iii) the reaction mixture after preparing the compound having formula (I) using the processes described in PCT International Application No. PCT/IB2020/058893. The entire content of each of WO2015/103144, WO2015/103142, and PCT/IB2020/058893 is hereby incorporated by reference.

### Isolation of Compound Having the Formula (I), Route 2

The present invention provides a method for isolating a compound having the formula (I) from a mixture comprising the compound having formula (I), DMS and at least one water immiscible solvent, wherein the method comprises (i) washing the mixture with an aqueous basic solution to form an organic phase and a water phase, (ii) separating the organic phase from the water phase, and (iii) crystallizing the compound having the formula (I) from the organic phase and filtering the crystals.

In some embodiments, the compound having formula (I) is a compound having formula (Iai):

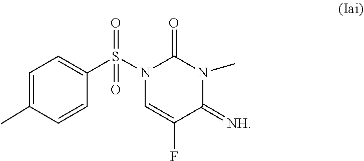

In some embodiments, the aqueous basic solution comprises DABCO, TBAB, NaOH, K₂CO₃, KHCO₃, Na₂CO₃, Et₃N, NaOMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof. In some embodiments, the aqueous basic solution is an aqueous solution of a base selected from the group consisting of K₂CO₃, KHCO₃, Na₂CO₃, NaHCO₃, K₂CO₃ NH₄OH, NaOH, and any combination thereof. In some embodiments, the aqueous basic solution is an aqueous solution of K₂CO₃.

In some embodiments, the concentration of the base in the aqueous basic solution is 2-18% based on the total weight (w/w). In some embodiments, the concentration of the base in the aqueous basic solution is about 15% based on the total weight (w/w).

In some embodiments, the compound having the formula (I) is dissolved in the water immiscible solvent or a mixture of a water immiscible solvent with a water miscible solvent to obtain an organic solution. In some embodiments, the compound having the formula (I) in salt form is dissolved in the water immiscible solvent or a mixture of a water immiscible solvent with a water miscible solvent to obtain an organic solution.

In some embodiments, the water immiscible solvent is polar. In some embodiments, the water immiscible solvent is non-polar. In some embodiments, the water immiscible solvent is an organic polar solvent.

In some embodiments, the water immiscible solvent includes but not limited to CPME, MeTHF, DCM, toluene, anisole or any combination thereof.

In some embodiments, the water immiscible solvent is selected from the group consisting of CPME, MeTHF, DCM, toluene, anisole, and any combination thereof.

In some embodiments, the water immiscible solvent is selected from the group consisting of methyl tetrahydrofuran (MeTHF), cyclopentylmethylether (CPME), toluene, anisole, and any mixture thereof.

In some embodiments, the water immiscible solvent is CPME. In some embodiments, the water immiscible solvent is MeTHF. In some embodiments, the water immiscible solvent is DCM. In some embodiments, the water immiscible solvent is toluene. In some embodiments, the water immiscible solvent is anisole.

In some embodiments, the compound having the formula (I) is crystalized from the organic phase. In some embodiments, the compound having the formula (I) is crystallized by concentrating the organic phase. In some embodiments, the compound having the formula (I) is crystallized by adding an anti-solvent. In some embodiments, the compound having the formula (I) is crystallized by seeding.

The present invention provides a method for isolating a compound having the formula (I) comprising (1) washing of an organic solution comprising a polar water immiscible solvent and a mixture of compound (I) and DMS with 2-18% w/w of aqueous basic solution, (2) separating the organic phase from the water phase, and (3) concentrating the organic phase and filtering the precipitated solid.

In some embodiments, the compound having formula (I) in the mixture is in salt form.

The present invention provides a method for isolating the compound having the formula (I) from a mixture comprising the compound having formula (I) and DMS, wherein the method comprises (1) dissolving the mixture comprising the compound having formula (I) and DMS in an organic polar solvent to obtain an organic solution, (2) washing the organic solution obtained from (1) with 2-18% w/w of aqueous basic solution, (3) separating the organic phase from the water phase, and (4) concentrating the organic phase and filtering the precipitated solid.

In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20.

In some embodiments, the organic polar solvent is an organic polar water immiscible solvent.

In some embodiments, the organic water immiscible solvent has a dielectric constant less than 20.

In some embodiments, organic water immiscible solvent includes but is not limited to methyl tetrahydrofuran (MeTHF), cyclopentylmethylether (CPME), and a mixture thereof.

In some embodiments, the organic polar solvent is selected from the group consisting of DMA, CPME, MeTHF, DMA, DMF, DCM and any combination thereof.

In some embodiments, the organic polar solvent is selected from the group consisting of CPME, MeTHF, DCM and any combination thereof.

In some embodiments, the organic polar solvent is DMA. In some embodiments, the organic polar solvent is CPME. In some embodiments, the organic polar solvent is MeTHF.

In some embodiments, the compound having formula (I) is a compound having formula (Ia) wherein R is alkyl.

In some embodiments, the compound having formula (Ia) is a compound having formula (Iai) wherein R is methyl.

In some embodiments, the organic phase is cooled prior to and/or during the filtration. In some embodiments, the organic phase is cooled to 0-5° C.

In some embodiments, the organic polar solvent is added after the reaction to obtain the compound having the formula (I). In some embodiments, the CPME is added after the reaction to obtain the compound having the formula (I).

In some embodiments, a partial amount of the organic polar solvent is present from the reaction to obtain the compound having formula (I) and optionally additional amount of the organic polar solvent is added before isolation of the compound having formula (I). In some embodiments, a partial amount of the CPME is present from the reaction to obtain the compound having formula (I) and optionally an additional amount of CPME is added before isolation of the compound having formula (I).

In some embodiments, the compound having the formula (I) in the mixture before the isolation step is in salt form.

In some embodiments, the non-polar solvent is a non-polar water immiscible solvent.

In some embodiments, the non-polar water immiscible solvent dissolves the compound of formula (I).

In some embodiments, the water immiscible solvent includes but is not limited to ether-based solvent, aromatic solvent such as CPME, THE, anisole, toluene, and any mixture thereof.

In some embodiments, the non-polar solvent is anisole. In some embodiments, the non-polar solvent is toluene.

In some embodiments, an anti-solvent is further added.

In some embodiments, the anti-solvent is added parallel to the to the water immiscible solvent.

In some embodiments, the anti-solvent is added dropwise.

In some embodiments, the crystallization is conducted at temperature less than 0° C.

In some embodiments, the anti-solvent is a C5-C11 alkane. In some embodiments, the anti-solvent is hexane. In some embodiments, the anti-solvent is heptane.

In some embodiments, the mixture of solvent and anti-solvent are anisole and hexane.

In some embodiments, the mixture of solvent and anti-solvent are toluene and hexane.

In some embodiments, the aqueous basic solution comprises DABCO, TBAB, NaOH, K₂CO₃, KHCO₃, Na₂CO₃, Et₃N, NaoMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof.

In some embodiments, the aqueous basic solution is an aqueous solution of K₂CO₃.

In some embodiments, the compound having formula (I) is a compound having formula (Ia) wherein R is alkyl.

In some embodiments, the compound having formula (Ia) is a compound having formula (Iai) wherein R is methyl.

In some embodiments, the organic phase is cooled prior to and/or during the filtration. In some embodiments, the organic phase is cooled to 0-5° C.

In some embodiments, the crystallization is done by concentration of the solvent.

In some embodiments, the crystallization is done with crystal seeding.

In some embodiments, the mixture is seeded with 0.1-1% of the compound of formula (I).

In some embodiments, the water immiscible solvent is added after the reaction to obtain the compound having the formula (I). In some embodiments, anisole and hexane are added after the reaction to obtain the compound having the formula (I).

In some embodiments, a partial amount of the water immiscible solvent is present from the reaction to obtain the compound having formula (I) and optionally additional amount of the water immiscible solvent is added before isolation of the compound having formula (I). In some embodiments, a partial amount of the anisole is present from the reaction to obtain the compound having formula (I) and optionally an additional amount of anisole optionally with hexane are added before isolation of the compound having formula (I).

The method for isolating a compound having formula (I) from a mixture thereof may be used to isolate a compound having formula (I) from any mixture thereof, including, but not limited to, (i) the reaction mixture after preparing the compound having formula (I) using the processes described herein, (ii) the reaction mixture after preparing the compound having formula (I) using the processes described in PCT International Application Publication Nos. WO2015/103144 and WO2015/103142, and (iii) the reaction mixture after preparing the compound having formula (I) using the processes described in PCT International Application No. PCT/IB2020/058893. The entire content of each of WO2015/103144, WO2015/103142, and PCT/IB2020/058893 is hereby incorporated by reference.

### Isolation of Compound Having the Formula (I), Route 3

The present invention provides a method for isolating a compound having the formula (I) from a mixture comprising the compound having formula (I), DMS, at least one water immiscible solvent, and at least one additional solvent, wherein the method comprises (i) washing the mixture with an aqueous basic solution to obtain slurry mixture comprising and solids, (ii) filtering the precipitated solids.

The present invention provides a method for isolating a compound having the formula (I) from a mixture comprising the compound having formula (I), DMS and at least one water immiscible solvent, wherein the method comprises (i) washing the mixture with an aqueous basic solution and at least one additional solvent to obtain slurry mixture comprising solids, and (ii) filtering the precipitated solids.

The present invention provides a method for isolating a compound having the formula (I) from a mixture comprising the compound having formula (I), DMS, and at least one solvent wherein the method comprises (i) washing the mixture with an aqueous basic solution to obtain a slurry mixture comprising solids, and (ii) filtering the precipitated solids.

In some embodiments, the compound having formula (I) is a compound having formula (Iai):

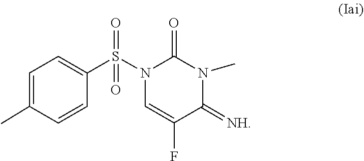

In some embodiments, the method includes adding additional solvent.

In some embodiments, the aqueous basic solution comprises DABCO, TBAB, NaOH, K₂CO₃, KHCO₃, Na₂CO₃, Et₃N, NaOMe, NaOEt or any combination thereof. In some embodiments, the aqueous basic solution is an aqueous solution of a base selected from the group consisting of DABCO, NaOH, K₂CO₃, KHCO₃, Na₂CO₃, Et₃N, NaOMe, NaOEt and any combination thereof. In some embodiments, the aqueous basic solution is an aqueous solution of a base selected from the group consisting of K₂CO₃, KHCO₃, Na₂CO₃, NaHCO₃, K₂CO₃ NH₄OH, NaOH, and any combination thereof. In some embodiments, the aqueous basic solution is an aqueous solution of K₂CO₃.

In some embodiments, the concentration of the base in the aqueous basic solution is 2-18% based on the total weight (w/w).

In some embodiments, the compound having the formula (I) in the mixture is in salt form.

In some embodiments, the mixture comprises a solvent. In some embodiments, the slurry mixture comprises a solvent.

The mixture comprising the compound having formula (I) may comprise DMS and any or all solvents used during the process for preparing the compound having formula (I).

The additional solvent is a solvent used to isolate or assist in the isolation of the compound having formula (I). The additional solvent may be added directly to the mixture. The additional solvent may also be added with the aqueous basic solution.

In some embodiments, the additional solvent is the same as the solvent(s) used during the process for preparing the compound having formula (I).

In some embodiments, the additional solvent is different from the solvent(s) used during the process for preparing the compound having formula (I).

In some embodiments, the additional solvent is a polar solvent.

In some embodiments, the additional solvent is a water immiscible solvent. In some embodiments, the polar solvent has a dielectric constant equal to or above 4. In some embodiments, the polar solvent has a dielectric constant equal to or above 5. In some embodiments, the polar solvent has a dielectric constant equal to or above 10. In some embodiments, the polar solvent has a dielectric constant equal to or above 15. In some embodiments, the polar solvent has a dielectric constant equal to or above 20. In some embodiments, the polar solvent has a dielectric constant of 4.7.

In some embodiments, the ratio between the additional solvent to the compound having formula (I) is greater than 0.5:1. In some embodiments, the ratio between the additional solvent to the compound having formula (I) is less than 2:1. In some embodiments, the ratio between the additional solvent to the compound having formula (I) is about 1:1.

In some embodiments, the ratio between the additional solvent to the compound having formula (II) used for preparing the compound having formula (I) is greater than 0.5:1. In some embodiments, the ratio between the additional solvent to the compound having formula (II) used for preparing the compound having formula (I) is less than 2:1. In some embodiments, the ratio between the additional solvent to the compound having formula (II) used for preparing the compound having formula (I) is about 1:1.

In some embodiments, the additional solvent is a polar solvent and the polar solvent is CPME.

In some embodiments, the solvent used during the process for preparing the compound having formula (I) is DMA and the additional solvent is CPME.

In some embodiments, the ratio between CPME to the compound having formula (I) is greater than 0.5:1. In some embodiments, the ratio between CPME to the compound having formula (I) is less than 2:1. In some embodiments, the ratio between CPME to the compound having formula (I) is about 1:1.

In some embodiments, the ratio between CPME to the compound having formula (II) used for preparing the compound having formula (I) is greater than 0.5:1. In some embodiments, the ratio between CPME to the compound having formula (II) used for preparing the compound having formula (I) is less than 2:1. In some embodiments, the ratio between CPME to the compound having formula (II) used for preparing the compound having formula (I) is about 1:1.

In some embodiments, the slurry mixture is mixed for 30 minutes to 8 hours.

In some embodiments, the slurry mixture is mixed at temperature between 25 to 60 degrees Celsius. In some embodiments, the slurry mixture is mixed at temperature between 25 to 50 degrees Celsius. In some embodiments, the slurry mixture is mixed at temperature between 25 to 35 degrees Celsius. In some embodiments, the slurry mixture is mixed at temperature of about 30 degrees Celsius.

In some embodiments, the slurry mixture is mixed using mechanical stirrer.

In some embodiments, the slurry mixture is mixed using high shear stirrer.

In some embodiments, the slurry mixture is mixed using both mechanical stirrer and high shear stirrer.

In some embodiments, the slurry mixture is obtained by adding the mixture comprising the compound having formula (I) and DMS and optionally organic solvent into a 2-18% of aqueous basic solution. In some embodiments, the slurry mixture is obtained by adding the mixture comprising the compound having formula (I) and DMS and optionally organic solvent into a 11-18% of aqueous basic solution.

In some embodiments, the slurry mixture is obtained by adding the mixture comprising the compound having formula (I) and DMS and optionally organic solvent into a 15% of aqueous basic solution.

In some embodiments, the slurry mixture obtained by adding the 2-18% aqueous basic solution into a mixture comprising the compound having formula (I), DMS and optionally an organic solvent.

In some embodiments, in step (2), the precipitated solids are filtered at a temperature between 15 to 45 degrees Celsius. In some embodiments, in step (2), the precipitated solids are filtered at a temperature between 15 to 20 degrees Celsius. In some embodiments, in step (2), the precipitated solids are filtered at a temperature between 20 to 25 degrees Celsius. In some embodiments, in step (2), the precipitated solids are filtered at a temperature between 25 to 30 degrees Celsius. In some embodiments, in step (2), the precipitated solids are filtered at a temperature between 30 to 35 degrees Celsius. In some embodiments, in step (2), the precipitated solids are filtered at a temperature between 35 to 40 degrees Celsius. In some embodiments, in step (2), the precipitated solids are filtered at a temperature between 40 to 45 degrees Celsius.

In some embodiments, the filtered solid obtained in step (2) is washed with an organic solvent during filtration in step (2). In some embodiments, the organic solvent is CPME.

In some embodiments, the filtered solid obtained in step (2) is washed with water during filtration in step (2).

In some embodiments, the filtered solid is mixed with water and stirred for 1 to 3 hours and filtered.

In some embodiments, the filtered solid is mixed with water and stirred at a temperature of 25-50 degrees Celsius and filtered.

In some embodiments, the organic solvent is the same organic solvent as used in obtaining the compound having formula (I).

In some embodiments, the aqueous basic solution is 15% of K₂CO₃ in water based on the total weight (w/w) of K₂CO₃ in water.

In some embodiments, the organic phase is the solution which is obtained in the reaction of compound having the formula (II) with DMS.

In some embodiments, the organic phase is obtained by adding an organic water immiscible solvent to the mixture of the compound having formula (I) and DMS obtained in the reaction of compound (II) and DMS.

In some embodiments, the step of adding an aqueous basic solution comprises additional use of a phase transfer catalyst (PTC) such as tetra-n-butylammonium bromide (TBAB).

In some embodiments, a solution of the compound having formula (I) in CPME is obtained by mixing CPME and the compound having formula (I) in weight ratio of 10:1 prior to washing with 2-18% w/w of aqueous basic solution.

In some embodiments, the solution of the compound having formula (I) in CPME is obtained by warming the combination of CPME and the compound having formula (I) up to 65° C. prior to washing with 2-18% w/w of aqueous basic solution.

In some embodiments, the solution of the compound having formula (I) in CPME is obtained by warming the combination of CPME and the compound having formula (I) up to about 50° C. prior to washing with 2-18% w/w of aqueous basic solution. In some embodiments, the resultant mixture obtained from the reaction of the compound having formula (II) with DMS is dissolved in CPME.

In some embodiments, the resultant mixture obtained from the reaction of the compound having formula (II) with DMS is dissolved with CPME and washed with water base solution.

In some embodiment the resultant mixture is a mixture of the compound having formula (I) with the solvent which was used in the reaction of the compound having formula (II) with DMS.

In some embodiments, the conversion of the 5-fluoro-4-imino-1-(toluene-4-sulfonyl)-3, 4-dihydro-1H-pyrimidin-2-one to 5-fluoro-4-imino-3-methyl-1-(toluene-4-sulfonyl)-3, 4-dihydro-1H-pyrimidin-2-one is higher than 50%.

In some embodiments, the chemical yield of 5-fluoro-4-imino-3-methyl-1-(toluene-4-sulfonyl)-3, 4-dihydro-1H-pyrimidin-2-one is higher than 50%. The yield of the purified 5-fluoro-4-imino-3-methyl-1-(toluene-4-sulfonyl)-3, 4-dihydro-1H-pyrimidin-2-one is higher than 508, 60%, 70%, 80%, 90% or 99%.

In some embodiments, the conversion of the 5-fluoro-4-imino-1-(toluene-4-sulfonyl)-3, 4-dihydro-1H-pyrimidin-2-one to 5-fluoro-4-imino-3-methyl-1-(phenyl-4-sulfonyl)-3,4-dihydro-1H-pyrimidin-2-one is higher than 50%.

In some embodiments, the chemical yield of 5-fluoro-4-imino-3-methyl-1-(phenyl-4-sulfonyl)-3, 4-dihydro-1H-pyrimidin-2-one is higher than 508. The yield of the purified 5-fluoro-4-imino-3-methyl-1-(toluene-4-sulfonyl)-3, 4-dihydro-1H-pyrimidin-2-one is higher than 50%, 60%, 70%, 80%, 90% or 99%.

The present invention provides a method for isolating a compound having the formula (I) from a mixture comprising the compound having formula (I) at least one water immiscible solvent, and at least one additional solvent, wherein the method comprises (i) washing the mixture with water to obtain slurry mixture containing solids, and (ii) filtering the precipitated solids.

The method for isolating a compound having formula (I) from a mixture thereof may be used to isolate a compound having formula (I) from any mixture thereof, including, but not limited to, (i) the reaction mixture after preparing the compound having formula (I) using the processes described herein, (ii) the reaction mixture after preparing the compound having formula (I) using the processes described in PCT International Application Publication Nos.

WO2015/103144 and WO2015/103142, and (iii) the reaction mixture after preparing the compound having formula (I) using the processes described in PCT International Application No. PCT/IB2020/058893. The entire content of each of WO2015/103144, WO2015/103142, and PCT/IB2020/058893 is hereby incorporated by reference.

The present invention also provides a compound having formula (I) prepared using the process described herein.

The present invention also provides a compound having formula (Iai) prepared using the process described herein.

The present reactions occur under reaction conditions sufficient to produce the desired compound. Such conditions, e.g. temperature, time, molarity, etc., may be varied by one of ordinary skill in the art based on the methods and protocols described herein.

The present invention also provides a method for crystallizing or recrystallizing a compound having formula (I), comprising (i) preparing a solution comprising a compound having formula (I) and a solvent, and (ii) contacting the solution with an anti-solvent.

In some embodiments, the anti-solvent is a C5-C11 alkane. In some embodiments, the anti-solvent is hexane. In some embodiments, the anti-solvent is heptane.

In some embodiment, the solvent is one where the anti-solvent can be dissolved in.

The method for crystallizing or recrystallizing a compound having formula (I) described herein may be used for crystallizing or recrystallizing the compound having formula (I) prepared using any process, including but not limited to the processes described herein and in PCT International Application Publication Nos. WO 2015/103142, WO 2015/103144, WO/2021/059160, and WO/2021/181274, the entire content of each of which is hereby incorporated by reference.

In some embodiments, the method comprises preparing the compound having formula (I) and crystallizing or recrystallizing the compound having formula (I) comprising (i) preparing a solution comprising the compound having formula (I) and a solvent, and (ii) contacting the solution with an anti-solvent.

The compound of having formula (I) may be prepared using any process, including but not limited to the processes described herein and in PCT International Application Publication Nos. WO 2015/103142, WO 2015/103144, WO/2021/059160 and WO/2021/181274, the entire content of each of which is hereby incorporated by reference.

The present invention also provides use of an anti-solvent to crystalize or recrystallize a compound having formula (I) from a solution thereof.

In some embodiments, the anti-solvent is a C5-C11 alkane. In some embodiments, the anti-solvent is hexane. In some embodiments, the anti-solvent is heptane.

In some embodiments, the compound having formula (I) is in a reaction mixture resulting from preparing the compound having formula (I) using any one of the processes described herein or any one of the processes described in PCT International Application Publication Nos. WO 2015/103142, WO 2015/103144, WO/2021/059160 and WO/2021/181274, the entire content of each of which is hereby incorporated by reference.

The present invention also provides a method for isolating a compound having formula (II) from a mixture comprising the compound having formula (II), wherein the method comprises (i) adding a protic solvent to the mixture to precipitate the compound having formula (II) from the mixture, and (ii) collecting the precipitated compound of formula (II).

In some embodiments, the protic solvent is water, methanol, or a combination thereof.

The methods for isolating a compound having formula (II) may be applied to any mixture comprising the compound having formula (II), including the reaction mixtures of the processes described herein for preparing the compound having formula (II) and the reaction mixtures of the processes described in PCT International Application Publication Nos. WO 2015/103142, WO 2015/103144, and WO/2021/181274 for preparing the compound having formula (II), the entire content of each of which is hereby incorporated by reference.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

The invention is illustrated by the following examples without limiting it thereby.

### EXAMPLES

### Examples of the Sulfonation Step

### Example 1: Reacting 5-Fluorocytosine with Compound Having the Formula (III) in DMA and Water and K₂CO₃ as Base

5-fluorocytosine (998, 80.1 gr) was added to 1 L glass reactor that contains 200 gr of water and 200 gr of DMA. K₂CO₃ (solid, 114.9 gr, 1.35 mol equiv.) was added and the reactor was cooled to 0° C. Tosyl chloride (128 gr, 1.05 mol equiv.) was added in one portion at 0° C. and the solution was mixed at temperature of 0° C. to 5° C. The reaction was monitored using HPLC and then 200 gr of water were added and the temperature was heated to 25° C. and mixed for 2 hours. The obtained solid was filtered off using Buchner funnel. The cake was washed with water and dried in vacuum oven at 55 to 65° C. 178 gr of desired product were obtained in purity of 90.9% and yield of 93%.

The product contains impurity A (2.4%) and impurity B (0.2%).

### Example 2: Reacting 5-Fluorocytosine with Compound Having the Formula (III) in DMA and Et₃N as Base

To 40 gr of 99.2% 5-fluorocytosine in 120 gr of DMA, 34.2 gr of Et₃N was added. The mixture cooled down to 0° C. 1.05 eq of TSCl was added, and the reaction mixture was stirred for 4 hours at 3° C. The reaction was monitored using HPLC. The conversion towards the product was 94.5%.

250 gr of water was added to the mixture and heated to 15° C. The solid was filtered off and dried in vacuum oven to obtain the desired product at 75% isolated yield. Chemical yield=77%. The product was obtained in 78.8% purity and contains impurity B (1%).

### Example 3: Reacting 5-Fluorocytosine with Compound Having the Formula (III) in DMA and Water and K₂CO₃ as Base

To 5 gr of 99.2% 5-fluorocytosine in 20 gr of water and 10 gr of DMA, 8.5 gr of K₂CO₃ (1.6 eq) was added. The reaction mixture was cooled down to 5° C., and 8.4 gr of TSCl (1.1 eq) was added. The reaction mixture was stirred for 3 hours at 5° C. HPLC monitoring detected 92% of the desired product. The precipitate was filtered off and dried in vacuum oven at 55° C. for 12 hours to yield the product as white solid with purity of 84% and isolated yield of 85%. The product contains impurity A (68).

### Example 4 (a): Reacting 5-Fluorocytosine with Compound Having the Formula (III) in ACN and Et₃N as Base

To 30 gr of 99.2% 5-fluorocytosine in 165 gr of ACN, 30.4 gr of Et₃N (1.3 eq) was added. The mixture was cooled down to 0° C. Afterwards, 50.6 gr of TSCl (1.15 eq) was added in two portions at 0° C. The reaction mixture was stirred for 4 hours. The reaction was monitored using HPLC. 310 gr of water was added to the mixture, heated to 10° C., and stirred for 1 hour. The solid was filtered off and dried in vacuum oven to obtain the desired product at 54% isolated yield. Chemical yield was 638. The product was obtained in purity of 68.8% and contains impurity B (6.1%) and impurity A (0.7%).

### Example 4 (b): Reacting 5-Fluorocytosine with Compound Having the Formula (III) in ACN and Et₃N as Base

To 5 gr of 99.2% 5-fluorocytosine in 15 gr of ACN, 4.7 gr of Et₃N was added. The mixture was cooled down to 5° C. Afterwards, 8.1 gr of TSCl was added, and the reaction mixture was stirred for 2 hours at 5° C. The reaction was monitored using HPLC. MeOH was added to the mixture and the solid was filtered off and dried in vacuum oven to obtain the desired product at 60.8% isolated yield. Chemical yield=74%. The product was obtained in purity of 73.7% and contains impurity B (0.38) and impurity A (0.5%).

### Example 4 (c): Reacting 5-Fluorocytosine with Compound Having the Formula (III) in MeCN and NEt₃

5-fluorocytosine (998, 5 gr) was added to a round bottom flask that contained 15 gr of acetonitrile. Triethylamine (4.6 gr, 1.2 mol equiv.) was added and the flask was cooled to 5° C. Tosyl chloride (8 gr, 1.1 mol equiv.) was added in one portion at 5° C. and the solution was mixed at a temperature of 5° C. The reaction was monitored using HPLC and then methanol was added and the temperature was heated to 25° C. and mixed for 2 hours. The obtained solid was filtered off using Buchner funnel. 9 gr of desired product was obtained in purity of 74% and yield of 618. The product contains impurity A (0.5%) and impurity B (0.3%).

### Example 5: Reacting 5-Fluorocytosine with Compound Having the Formula (III) in Water and DMA and DMAP as Base

To 5 gr of 99.2% of 5-fluorocytosine in 15 gr of water and 10 gr of DMA, 1.4 gr of DMAP was added. The reaction mixture was cooled down to 5° C., and 9.15 gr of TSCl was added. The reaction pH was adjusted to 9-10, using 20% of NaOH solution. After reaching 868 of the product in HPLC, the reaction mixture was heated to 25° C. The precipitate was filtered off, washed with water and dried in vacuum oven to yield the desired product in isolated yield of 59%.

The product was obtained in purity of 79.6% and contains impurity B (0.75%) and impurity A (0.32%).

### Example 6: Reacting 5-Fluorocytosine with Compound Having the Formula (III) in MeCN and Triethylamine as Base

To 30 gr of 99.2% 5-fluorocytosine in 165 gr of ACN, 30.4 gr of Et₃N (1.3 eq) was added. The mixture was cooled down to 0° C. Afterwards, 50.6 gr of TSCl (1.15 eq) was added in two portions at 0° C. The reaction mixture was stirred for 4 hours. The reaction was monitored using HPLC. 310 gr of water was added to the mixture, heated to 10° C., and stirred for 1 hour. The solid was filtered off and dried in vacuum oven to obtain the desired product at 54% isolated yield. Chemical yield was 63%. The product was obtained in purity of 68.8% and contains impurity B (6.1%) and impurity A (0.7%).

### Example 7: Reacting 5-Fluorocytosine with Compound Having the Formula (III) in MeCN and Triethylamine as Base

To 5 gr of 99.2% 5-fluorocytosine in 15 gr of MeCN, 4.7 gr of Et₃N was added. The mixture was cooled down to 5° C. Afterwards, 8.1 gr of TSCl was added, and the reaction mixture was stirred for 2 hours at 5° C. The reaction was monitored using HPLC. MeOH was added to the mixture and the solid was filtered off and dried in vacuum oven to obtain the desired product at 60.8% isolated yield. Chemical yield=748. The product was obtained in purity of 73.7% and contains impurity B (0.3%) and impurity A (0.5%).

### Example 8: Reacting 5-Fluorocytosine with Compound Having the Formula (III) in DMA and Sodium Carbonate as Base

To 5 gr of 99.2% 5-fluorocytosine in 35 gr of DMA, 6.38 gr of Na₂CO₃ is added. The mixture is cooled down to 5° C. Afterwards, 8.37 gr of TSCl is added, and the reaction mixture is stirred for 3 hr at 5° C. The reaction is sampled in HPLC. Partial conversion of starting material is obtained. The product is not isolated.

### Example 9: Reacting 5-Fluorocytosine with Compound Having the Formula (III) in DMBA and Sodium Carbonate as Base

To 5 gr of 99.2% 5-fluorocytosine in 35 gr of DMBA, 6.38 gr of Na CO₃ is added. The mixture is cooled down to 5° C. Afterwards, 8.37 gr of TSCl is added, and the reaction mixture is stirred for 3 hr at 5° C. The reaction is sampled in HPLC. Partial conversion of starting material is obtained. The product is not isolated.

### Example 10: Reacting 5-Fluorocytosine with Compound Having the Formula (III) in Water and DMAP as Base

To 5 gr of 99.2% 5-fluorocytosine in 40 gr of water, 2.35 gr of 4-DMAP was added. 8.8 gr of TSCl was added, and the reaction mixture stirred for 4 hr at 25° C. The reaction was sampled in HPLC. 50% of desired product was obtained. The conversion of starting material was 518. The product was not isolated.

### Example 11: Reacting 5-Fluorocytosine with Compound Having the Formula (III) in DMA, Water and DMBA and Potassium Hydroxide as Base

To 5 gr of 99.2% 5-fluorocytosine in 15 gr of DMA, 15 gr of water and 1.56 gr of N, N-dimethylbenzylamine are added. The mixture is cooled down to 5° C. Afterwards, 9.13 gr of TSCl is added, and the reaction mixture is stirred for 4 hr at 5° C. while keeping pH at 9-10 using potassium hydroxide 20% in water. The reaction is sampled in HPLC. The product is obtained in partial selectivity and is not isolated.

### Example 12: Reacting 5-Fluorocytosine with Compound Having the Formula (III) in DMA, Water and DMBA and Sodium Hydroxide as Base

To 5 gr of 99.2% 5-fluorocytosine in 15 gr of DMA, 15 gr of water and 1.56 gr of N, N-dimethylbenzylamine was added. The mixture was cooled down to 5° C. Afterwards, 9.13 gr of TSCl was added, and the reaction mixture was stirred for 4 hr at 5° C. while keeping pH at 9-10 using sodium hydroxide 20% in water. The reaction was sampled in HPLC. 78.3% of desired product was obtained, in conversion of 82%. 3.8% of product isomer was obtained. The product was not isolated.

### Example 13: Reacting 5-Fluorocytosine with Compound Having the Formula (III) in DMA and Water and K₂CO₃ as Base

5-fluorocytosine (998, 120 gr) was added to 1 L glass reactor that contains 360 gr water and 160 gr of DMA. K₂CO₃ (204 gr, 1.6 mol equiv.) was added and the reactor was cooled to −5° C. Tosyl chloride (192 gr, 1.05 mol equiv.) was added in one portion at −5° C. to −3° C. over 1.5 hours and the solution was mixed at temperature of −5° C. for 3.5 hours. The reaction was monitored using HPLC. 93% selectivity was observed. The reaction was heated to 15° C. and the product was filtered off and dried in vacuum oven at 55° C. 329 gr of product in purity of 60% was obtained. The isolated yield was 76%.

### Examples of the Alkylation Step

### Example 14: Reaction of Compound Having the Formula (IIai) with Dimethylsulphate (DMS) in Absence of Base in CPME and DMA

4800 gr CPME, 1200 gr DMA and 1700 gr 5-fluoro-4-imino-1-(toluene-4-sulfonyl)-3, 4-dihydro-1H-pyrimidin-2-one (IIai) (81% purity) were added into a 25 L reactor. The mixture was heated to 35° C. and dimethylsulfate was added (2000 gr, 3.5 mol equiv.) over 30 minutes. The reaction was stirred for 4 hours at 35-40° C. 9000 gr CPME was added and the mixture was heated to 50° C. until a clear solution was obtained. An aqueous solution of K₂CO₃ (10% w, 10 kg) was added and the mixture was stirred for 30 minutes. The phases were separated and the organic phase was mixed with another 5 kg solution of 10% K₂CO₃ containing 85 gr of TBAB for 1 hour followed by phase separation and repeating the same procedure again. The organic phase was washed with 9 kg of water and then 10 kg of CPME was evaporated at 100 mbar at 50° C.

The solution was cooled to 0-5° C. and the obtained solid was filtered off using Buchner funnel. The cake was washed with 1 liter of cold water and dried in vacuum oven at 65° C. 1222 gr of product was obtained in 98.9% purity and 80% isolated yield.

### Example 15 (a): Reaction of Compound Having the Formula (IIai) with Dimethylsulphate (DMS) in CPME and DMA and in the Presence of TBAB

To a mixture of 40 gr of 5-fluoro-4-imino-1-(toluene-4-sulfonyl)-3, 4-dihydro-1H-pyrimidin-2-one (IIai) at purity of 86.8%, 200 gr of CPME, 50 gr of DMA, TBAB 5%, and 3 eq of dimethylsulfate was added as one portion. The reaction mixture was heated to 40° C. for 6 hours. Then, 250 gr of CPME was added, and the reaction mixture heated to 50° C. and washed twice with 300 gr of 10% K₂CO₃ solution. The last wash was with 300 gr of water at 50° C. The organic phase was concentrated under reduced pressure and the residues cooled down to 5° C., the formed solid was filtered off, washed with CPME and dried in vacuum oven at 65° C. to obtain the desired product at 94.5% purity, and isolated yield of 80.4%.

### Example 15 (b): Reaction of Compound Having the Formula (IIai) with Dimethylsulphate (DMS) in DMA and in the Presence of NaOMe

To a mixture of 5 gr 5-fluoro-4-imino-1-(toluene-4-sulfonyl)-3, 4-dihydro-1H-pyrimidin-2-one (IIai) at purity of 85.38, 15 gr of DMA, 0.2 eq of NaOMe, 4 eq of dimethylsulfate was added. The reaction mixture was heated to 35° C. for 5 hours. 45 gr of CPME was added, and heated to 50° C. The organic phase was washed twice using 10% K₂CO₃ solution, and washed a third time with 50 gr of water. The phase separation was performed at 50° C. The organic phase was concentrated under vacuum to yield the desired product at 51% isolated yield.

### Example 16: Reaction of Compound Having the Formula (IIai) with Dimethylsulphate (DMS) in Absence of Base in CPME and DMA 1:1

2000 gr CPME, 2000 gr DMA and 2000 gr 5-fluoro-4-imino-1-(toluene-4-sulfonyl)-3, 4-dihydro-1H-pyrimidin-2-one (IIai) (81% purity) were added into a 25 L reactor. The mixture was heated to 35° C. and dimethylsulfate was added (2200 gr, 3 mol equiv.) over 60 minutes. The reaction was stirred for 4 hours at 35-40° C. 14000 gr CPME was added and the mixture was heated to 50° C. until a clear solution was obtained. An aqueous solution of K₂CO₃ (15% w, 11.5 kg) was added and the mixture was stirred for 60 minutes at 60° C. The phases were separated and the organic phase was mixed with another 6 kg solution of 10% K₂CO₃ for 0.5 hour followed by phase separation. The organic phase was stirred for 30 minutes with 6.3 kg water and the phases were separated. 11 kg CPME was evaporated at 100 mbar at 50° C.

The solution was cooled to 0-5° C. and the obtained solid was filtered off using Buchner funnel. The cake was washed with 1 liter of cold water and dried in vacuum oven at 65° C. The product was obtained in 75% isolated yield.

### Example 17: Reaction of Compound Having the Formula (IIai) with Dimethylsulphate (DMS) in Absence of Base in DMA

200 gr DMA and 100 gr 5-fluoro-4-imino-1-(toluene-4-sulfonyl)-3, 4-dihydro-1H-pyrimidin-2-one (IIai) (818 purity) were added into a 25 L reactor. The mixture was heated to 35° C. and dimethylsulfate was added (110 gr, 2.5 mol equiv.) over 60 minutes. The reaction was stirred for 4 hours at 35-40° C. 50 gr CPME and aqueous solution of K₂CO₃ (15% w, 600 gr) was added and the mixture was heated to 60° C. was stirred for 120 minutes at 45° C.

The mixture was cooled to 0-5° C. over 1 hour and the obtained solid was filtered off using Buchner funnel. The cake was mixed with 300 gr water for 1 hour at 40° C., cooled to 0° C., filtered and the process of water slurry repeated again. The product was dried in vacuum oven at 65° C. The product was obtained in 80% isolated yield in 99% purity.

### Example 18: Reaction of Compound Having the Formula (IIai) with Dimethylsulphate (DMS) 5 Equiv. in Absence of Base in DMA and CPME

28 gr CPME, 7 gr DMA and 10 gr 5-fluoro-4-imino-1-(toluene-4-sulfonyl)-3, 4-dihydro-1H-pyrimidin-2-one (81% purity) were added into a round bottom flask. The mixture was heated to 35° C. and dimethylsulfate was added (18 gr, 5 mol equiv.) over 30 minutes. The reaction was stirred for 5.5 hours at 39° C. 53 gr CPME was added and the mixture was heated to 58° C. until a clear solution was obtained. An aqueous solution of K₂CO₃ (10% w) was added and the mixture was stirred for 30 minutes. The phases were separated and the organic phase was mixed with another 176 gr solution of 10% K₂CO₃ containing 0.5 gr of TBAB for 1 hour followed by phase separation and repeating the same procedure again. The organic phase was washed with 300 gr of water and then 300 gr of CPME was evaporated at 100 mbar at 50° C.

The solution was cooled to 0-5° C. and the obtained solid was filtered off using Buchner funnel. The cake was washed with 1 liter of cold water and dried in vacuum oven at 65° C. The product was obtained in 99.5% purity and 81% isolated yield.

### Example 19: Reaction of Compound Having the Formula (IIai) with Dimethylsulphate (DMS) in Presence of Base in DMA and CPME

5.6 gr CPME, 1.4 gr DMA and 2 gr 5-fluoro-4-imino-1-(toluene-4-sulfonyl)-3, 4-dihydro-1H-pyrimidin-2-one (81% purity) were added into a round bottom flask. The mixture was heated to 35° C. and dimethylsulfate was added (2.5 gr, 3.5 mol equiv.) over 30 minutes. The reaction was stirred for 4 hours at 40° C. After 4 hours, Na₂CO₃ (0.3 equiv.) was added and the stirring was continued for an additional hour until complete conversion of starting material.

10 gr CPME was added and the mixture was heated to 56° C. until a clear solution was obtained. An aqueous solution of K₂CO₃ (10% w) was added and the mixture was stirred for 30 minutes. The phases were separated and the organic phase was mixed with another 35 gr solution of 10% K₂CO₃ for 1 hour followed by phase separation and repeating the same procedure again. The organic phase was washed with 60 gr of water and then 60 gr of CPME was evaporated at 100 mbar at 50° C.

The solution was cooled to 0-5° C. and the obtained solid was filtered off using Buchner funnel. The cake was washed with 1 liter of cold water and dried in vacuum oven at 65° C. The product was obtained in 97.5% purity and 74% isolated yield.

### Example 20: Reaction of Compound Having the Formula (IIai) with Dimethylsulphate (DMS) in Presence of Base in DMA and CPME

28 gr CPME, 7 gr DMA and 10 gr 5-fluoro-4-imino-1-(toluene-4-sulfonyl)-3, 4-dihydro-1H-pyrimidin-2-one (818 purity) were added into a round bottom flask. The mixture was heated to 35° C. and dimethylsulfate was added (12.6 gr, 3.5 mol equiv.) over 30 minutes. The reaction was stirred for 4 hours at 39° C. After 4 hours, NaOMe (0.5 equiv) was added and the stirring was continued for an additional hour until complete conversion of starting material. 53 gr CPME was added and the mixture was heated to 58° C. until a clear solution was obtained. An aqueous solution of K₂CO₃ (10% w) was added and the mixture was stirred for 30 minutes. The phases were separated and the organic phase was mixed with another 176 gr solution of 10% K₂CO₃ containing 0.5 gr of TBAB for 1 hour followed by phase separation and repeating the same procedure again. The organic phase was washed with 300 gr of water and then 300 gr of CPME was evaporated at 100 mbar at 50° C.

The solution was cooled to 0-5° C. and the obtained solid was filtered off using Buchner funnel. The cake was washed with 1 liter of cold water and dried in vacuum oven at 65° C. The product was obtained in 98.8% purity and 77% isolated yield.

### Example 21: Reaction of Compound Having the Formula (IIai) with Dimethylsulphate (DMS) in High Shear Stirring in DMA/CPME

200 gr CPME, 200 gr DMA and 200 gr 5-fluoro-4-imino-1-(toluene-4-sulfonyl)-3, 4-dihydro-1H-pyrimidin-2-one (81% purity) were added into a reactor. The mixture was heated to 35° C. and dimethylsulfate was added (266 gr, 3.5 mol equiv.) over 30 minutes. The reaction was stirred for 5.5 hours at 35-40° C. 1/10 of the reaction mixture were taken into a 250 mL reactor equipped with high shear stirrer. 60 gr of CPME was added and the mixture was heated to 60° C. 120 gr aqueous solution of potassium carbonate 15% was added over 10 minutes. The mixture was cooled to 50° C. and the mixture was stirred for 2 hours followed by additional stirring for 2 hours at 30° C. The mixture was cooled to 0° C., filtered using buchner funnel and the cake was dried at 65° C. overnight in vacuum oven. The product was obtained in 97.7% purity and 62% yield.

### Example 22: Reaction of Compound Having the Formula (IIai) with Dimethylsulphate (DMS) in DMA and CPME

200 gr CPME, 200 gr DMA and 200 gr 5-fluoro-4-imino-1-(toluene-4-sulfonyl)-3, 4-dihydro-1H-pyrimidin-2-one (81% purity) were added into a reactor. The mixture was heated to 35° C. and dimethylsulfate was added (266 gr, 3.5 mol equiv.) over 30 minutes. The reaction was stirred for 5.5 hours at 35-40° C. 193 gr of the reaction mixture were taken into a 1 L reactor equipped with mechanical stirrer.

151 gr of CPME was added and the mixture was heated to 60° C. over 10 minutes. 300 gr aqueous solution of potassium carbonate 15% was added over 20 minutes. The mixture was cooled to 30° C. and the mixture was stirred for 6 hours followed by cooling to 0° C. over 15 minutes. The product was filtered using buchner funnel and the cake was washed with 200 gr water and dried at 65° C. overnight in vacuum oven. The product was obtained in 98.1% purity and 60% yield.

### Example 23: Reaction of Compound Having the Formula (IIai) with Dimethylsulphate (DMS) in DMA and CPME

200 gr CPME, 200 gr DMA and 200 gr 5-fluoro-4-imino-1-(toluene-4-sulfonyl)-3, 4-dihydro-1H-pyrimidin-2-one (81% purity) were added into a reactor. The mixture was heated to 35° C. and dimethylsulfate was added (266 gr, 3.5 mol equiv.) over 30 minutes. The reaction was stirred for 5.5 hours at 35-40° C. The mixture was heated to 60° C. over 10 minutes and added dropwise into a reactor containing potassium carbonate 15% solution. The mixture was cooled to 30° C. and the mixture was stirred for 3 hours followed by cooling to 0° C. over 1.5 hours. The product was filtered using buchner funnel and the cake was washed with 150 gr water and filtered. the wet cake was mixed with 700 gr of water for 3 hours, filtered and washed with 150 gr water, filtered and dried in vacuum oven at 65° C. The product was obtained in 97% purity and 65% yield.

### Example 24: Reaction of Compound Having the Formula (IIai) with Dimethylsulphate (DMS) in CPME at 85°

15 gr CPME and 2 gr 5-fluoro-4-imino-1-(toluene-4-sulfonyl)-3, 4-dihydro-1H-pyrimidin-2-one (90.7% purity) were added into a round bottom flask. Dimethylsulfate was added (6 gr, 7.4 mol equiv.) in one portion. The reaction was stirred for 3 hours at 85° C. 53% selectivity and 81% conversion of starting material was observed according to HPLC. The product was not isolated.

### Example 25: Reaction of Compound Having the Formula (IIai) with Dimethylsulphate (DMS) in CPME/DMA at 25° C.

6 gr CPME, 2 gr DMA and 5 gr 5-fluoro-4-imino-1-(toluene-4-sulfonyl)-3, 4-dihydro-1H-pyrimidin-2-one (90.7% purity) were added into a round bottom flask. Dimethylsulfate was added (6 gr, 3 mol equiv.) in one portion. 2 gr 78 NaOH solution was added and the reaction was stirred at room temperature for 6 hours. 60% selectivity and 84% conversion of starting material was observed according to HPLC. The product was not isolated.

### Example 26: Reaction of Compound Having the Formula (IIai) with Dimethylsulphate (DMS) in MeTHF Using Triethylamine as Base

10 gr Methyltetrahydrofurane and 5 gr 5-fluoro-4-imino-1-(toluene-4-sulfonyl)-3, 4-dihydro-1H-pyrimidin-2-one (90.7% purity) and 0.8 gr triethylamine were into a round added bottom flask. Dimethylsulfate was added (4 gr, 5 mol equiv.) in one portion. The reaction was stirred at 45° C. for 6 hours. 73% selectivity and 81% conversion of starting material was observed according to HPLC. The product was not isolated.

### Example 27: Reaction of Compound Having the Formula (IIai) with Dimethylsulphate (DMS) 10 Mol Equiv. in MeTHF

15 gr Methyltetrahydrofurane and 2 gr 5-fluoro-4-imino-1-(toluene-4-sulfonyl)-3, 4-dihydro-1H-pyrimidin-2-one (90.7% purity) were added into a round bottom flask. Dimethylsulfate was added (8 gr, 10 mol equiv.) in one portion. The reaction was stirred at 40° C. for 5 hours. 62% selectivity and 70% conversion of starting material was observed according to HPLC. The product was not isolated.

### Example 28: Reaction of Compound Having the Formula (IIai) with Dimethylsulphate (DMS) in DMA and Anisole

To 57.1 gr 4-Amino-5-fluoro-1-[(4-methylphenyl) sulfonyl]-2 (1H)-pyrimidinone (87.5% purity), 50 gr of DMAA and 50 gr of anisole added at room temperature.

The reaction mixture was heated to Tr=35° C.

Dimethyl sulfate (55.7 gr 2.5Eq) was added dropwise at 30-60 min.

The reaction mixture was heated to Tr=40° C. and stirred for 3 hr.

50 gr of n-Hexane and 150 gr of Anisole added.

50 gr of K₂CO₃₍ₛ₎ were added to the reactor along with 300 gr water.

The reactor mixture was heated to 60° C.

After the stirring, the lower phase was discarded, and the upper phase was cooled to 30° C. and seeded with 1% of the compound of formula (I).

This mixture was mixed for 30 min at 30° C. then cooled to 0° C. during 3 hr.

The reaction mixture was stirred O.N and filtered.

The cake was washed with 50 gr n-Hexane and then with water Wet cake dried under vacuum at 65° C.

### DISCUSSION

There is a need to develop an improved synthetic process for producing 5-(fluoro-4-imino-3-methyl)-1-tosyl-3, 4-dihydro-pyrimidine-(1 h)-one.

The claimed process is an improvement over the processes described in WO2015/103144 and/or WO2015/103142 for synthesizing 5-(fluoro-4-imino-3-methyl)-1-tosyl-3, 4-dihydro-pyrimidine-(1 h)-one.

The present invention provides an efficient pathway for synthesis of compound I with two step reaction wherein each step is one step reaction without additional protection group and/or without using the alkylation reaction as described in WO2015/103144 and/or WO2015/103142.

The process is designed to solve the problem of non-selective sulfonation and alkylation steps as described in the previous process.

### REFERENCES

WO 2015/103144, published Jul. 9, 2015 (Adama Makhteshim Ltd.)
WO 2015/103142, published Jul. 9, 2015 (Adama Makhteshim Ltd.)

1. A process for obtaining 5-fluoro-4-imino-3-methyl-1-(phenyl-4-sulfonyl)-3, 4-dihydro-1H-pyrimidin-2-one having formula (I): 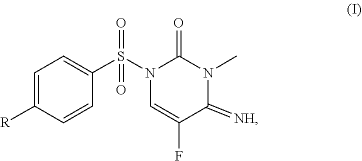
comprising:
(1) preparing the compound having formula (I) by reacting a compound having the formula (II) 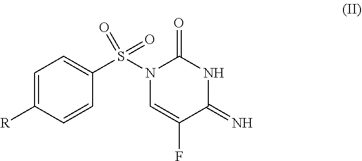
with dimethylsulphate (DMS), wherein R is hydrogen, alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃, and
(2) isolating the compound having formula (I) from the reaction mixture by adding an aqueous basic solution to the reaction mixture and obtaining the compound having formula (I),
wherein the process comprises use of at least one water immiscible solvent, wherein:
i) step (1) is conducted in the presence of at least one water-immiscible solvent, and/or
ii) at least one water-immiscible solvent is added to the reaction mixture after step (1).

2. The process of claim 1, wherein:
a) R is alkyl, alkenyl, alkynyl, alkoxy, haloalkyl, haloalkoxy, alkylthio, haloalkylthio, amino, alkylamino, dialkylamino, alkoxycarbonyl, alkyl carbonyl, hydroxyalkyl, ester, acid halogen, —SH, —OH, —NH₂, —NO₂, —CN or CF₃,
b) the water immiscible solvent is an ether-based solvent, an aromatic solvent, or a mixture thereof,
c) the aqueous basic solution comprises DABCO, TBAB, NaOH, K₂CO₃, KHCO₃, Na₂CO₃, Et₃N, NaOMe, NaOEt or any combination thereof,
d) the concentration of the base in the aqueous basic solution is 2-18% based on the total weight (w/w), and/or
e) the compound having formula (I) is in salt form.

3. (canceled)

4. The process of claim 1, wherein a multi-phase system is obtained and wherein:
a) the multi-phase system comprises an organic phase and a water phase and the process of isolating the compound having formula (I) from the reaction mixture comprises separating the organic phase from the water phase, crystallizing the compound having formula (I) from the organic phase, and filtering the crystals,
b) the multi-phase system is a slurry comprising solids, the process for isolating the compound having formula (I) form the reaction mixture comprises filtering the precipitated solids, or
c) the multi-phase system comprises a liquid and solids and the process for isolating the compound having formula (I) from the reaction mixture comprises filtering the solids.

5. (canceled)

6. (canceled)

7. (canceled)

8. (canceled)

9. The process of claim 1, wherein:
a) the water-immiscible solvent is toluene, anisole, or a mixture thereof,
b) the aqueous basic solution is an aqueous solution of K₂CO₃,
c) the molar ratio between the solvent(s) to the compound having formula (II) is between 30:1 and 1:1,
d) the molar ratio between the solvent(s) to DMS is between 10:1 to 1:1,
e) the molar ratio between the compound having formula (II) and DMS is between 1:2 to 1:10,
f) the reaction of the compound having formula (II) with DMS is conducted at a temperature between 25-85° C., and/or
g) the reaction of the compound having formula (II) with DMS to obtain the compound having formula (I) has a yield of at least 60%.

10. (canceled)

11. The process of claim 1, wherein:
a) the water immiscible solvent is added after step (1) and before addition of the aqueous basic solution to the reaction mixture, the water immiscible solvent is added at the same time as addition of the aqueous basic solution to the reaction mixture, or the water immiscible solvent is added after addition of the aqueous basic solution to the reaction mixture, or
b) the reaction is conducted in the presence of a mixture of solvents wherein at least one solvent is water immiscible and at least one solvent is water miscible.

12. (canceled)

13. The process of claim 11, wherein the mixture of solvents is DMA and anisole.

14. The process of claim 13, wherein:
a. the weight ratio between DMA and anisole is between 100:1 to 1:1,
b. the weight ratio between DMA and the compound having formula (II) is from about 15:1 to about 0.5:1, and/or
c. the weight ratio between anisole and the compound having formula (II) is from about 10:1 to about 1:1.

15. The process of claim 11, wherein the mixture of solvents is a mixture of DMA and toluene.

16. The process of claim 15, wherein:
a. the weight ratio between DMA and toluene is between 100:1 to 1:1,
b. the weight ratio between DMA and the compound having formula (II) is from about 15:1 to about 0.5:1, and/or
c. the weight ratio between toluene and the compound having formula (II) is from about 10:1 to about 1:1.

17. (canceled)

18. The process of claim 1, wherein the compound having formula (I) is in salt form and the salt is a monomethylsulfate salt of the compound having formula (I).

19. The process of claim 1, wherein the process comprises:
(a) (1) preparing the compound having formula (I) by reacting the compound having the formula (II) with dimethylsulphate (DMS) in the presence of at least one water immiscible solvent, and (2) isolating the compound having formula (I) from the reaction mixture by (i) washing the reaction mixture with an aqueous basic solution to from an organic phase and a water phase, (ii) separating the organic phase from the water phase, and (iii) crystalizing the compound having the formula (I) from the organic phase and filtering the crystals,
(b) (1) preparing the compound having formula (I) by reacting the compound having the formula (II) with dimethylsulphate (DMS), (2) adding at least one water immiscible solvent to the reaction mixture, and (3) isolating the compound having formula (I) from the reaction mixture by (i) washing the reaction mixture with an aqueous basic solution to form an organic phase and a water phase, (ii) separating the organic phase from the water phase, and (iii) crystalizing the compound having the formula (I) from the reaction mixture and filtering the crystallized solid, or
(c) (1) preparing the compound having formula (I) by reacting the compound having the formula (II) with dimethylsulphate (DMS), and (2) isolating the compound having formula (I) from the reaction mixture by (i) adding at least one water immiscible solvent and an aqueous basic solution to the reaction mixture to form an organic phase and a water phase, (ii) separating the organic phase from the water phase, and (iii) crystalizing the compound having the formula (I) and filtering the crystallized solid.

20. The process claim 1, wherein the process comprises:
(a) (1) preparing the compound having formula (I) by reacting the compound having the formula (II) with dimethylsulphate (DMS) in the presence of at least one water immiscible solvent and at least one additional solvent, and (2) isolating the compound having formula (I) from the reaction mixture by (i) washing the reaction mixture with an aqueous basic solution to obtain a slurry mixture comprising precipitated solids, and (ii) filtering the precipitated solids,
(b) (1) preparing the compound having formula (I) by reacting the compound having the formula (II) with dimethylsulphate (DMS), (2) adding at least one water immiscible solvent and at least one additional solvent to the reaction mixture, and (3) isolating the compound having formula (I) from the reaction mixture by (i) washing the reaction mixture with an aqueous basic solution to obtain a slurry mixture containing precipitated solids, and (ii) filtering the precipitated solids,
(c) (1) preparing the compound having formula (I) by reacting the compound having the formula (II) with dimethylsulphate (DMS), (2) adding at least one water immiscible solvent to the reaction mixture, and (3) isolating the compound having formula (I) from the reaction mixture by (i) washing the reaction mixture with an aqueous basic solution and at least one additional solvent to obtain a slurry mixture containing precipitated solids, and (ii) filtering the precipitated solids, or
(d) (1) preparing the compound having formula (I) by reacting the compound having the formula (II) with dimethylsulphate (DMS), and (2) isolating the compound having formula (I) from the reaction mixture by (i) adding an aqueous basic solution, at least one water immiscible solvent and at least one additional solvent to obtain a slurry mixture containing precipitated solids, and (ii) filtering the precipitated solids.

21. (canceled)

22. (canceled)

23. (canceled)

24. (canceled)

25. The process of claim 1, wherein the compound having formula (I) is a compound having formula (Iai): 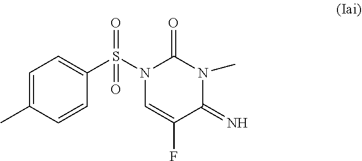
and the compound having formula (II) is a compound having formula (IIai) 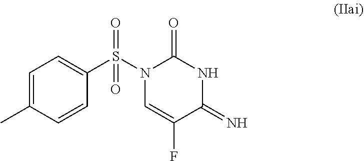
wherein R is methyl and X is a halogen or —OSO₂PhR.

26. A compound having formula (I) obtained using the process of claim 1.

27. A process for obtaining the compound 5-fluoro-4-imino-3-methyl-1-(phenyl-4-sulfonyl)-3, 4-dihydro-1H-pyrimidin-2-one having formula (I): 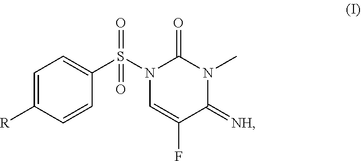
comprising:
(a) reacting 5-fluorocytosine with compound having formula (III): 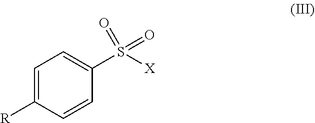
in the presence of at least one polar solvent and at least one base to obtain the compound having formula (II) 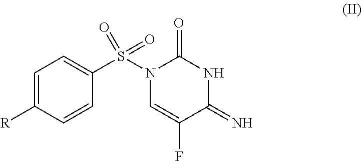
and
(b) preparing the compound having formula (I) and isolating the compound having formula (I) from the reaction mixture in accordance with the process of claim 1.

28. (canceled)

29. (canceled)

30. A method for isolating a compound having formula (I) from a mixture comprising the compound having formula (I), wherein the method comprises (i) preparing a multi-phase system comprising the compound having formula (I), a water-immiscible solvent and water, and (ii) obtaining and isolating solids of the compound having formula (I) from the multi-phase system.

31. (canceled)

32. The method of claim 30, wherein the compound having formula (I) is a compound having formula (Iai):

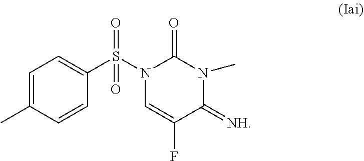

33. A monomethylsulfate salt of the compound having formula (I).

34. A method for crystallizing or recrystallizing a compound having formula (I), comprising (i) preparing a solution comprising a compound having formula (I) and a solvent, and (ii) contacting the solution with an anti-solvent.

35. (canceled)

36. A method for isolating a compound having formula (II) from a mixture comprising the compound having formula (II), wherein the method comprises (i) adding a protic solvent to the mixture to precipitate the compound having formula (II) from the mixture, and (ii) collecting the precipitated compound of formula (II).
